(19) United States
(12) Patent Application Publication
(10) Pub. No.: US 2009/0137991 A1
(43) Pub. Date: May 28, 2009

(54) Methods and Apparatus for Laser Treatment of the Crystalline Lens

(76) Inventor: Ronald M. Kurtz, Irvine, CA (US)

Correspondence Address: FISH & RICHARDSON, PC, P.O. BOX 1022, MINNEAPOLIS, MN, 55440-1022, US

(21) Appl. No.: 12/233,518

(22) Filed: Sep. 18, 2008

Related U.S. Application Data
(60) Provisional application No. 60/973,411, filed on Sep. 18, 2007.

(51) Int. Cl. A61F 9/008

(52) U.S. Cl. 606/5; 606/6

(57) ABSTRACT

Methods and apparatus for laser treatment of the crystalline lens. Implementations of the described methods and apparatus include a laser treatment of a lens of an eye includes defining a target boundary of a target region in the lens, applying surgical laser pulses to the target boundary effectively resulting in a separation of the target region from the rest of the lens, and removing the separated target region from the lens. The target boundary can be defined by applying marker laser pulses to outline the target boundary. The marker laser pulses can be applied by a laser source using marker pulse settings and the surgical laser pulses can be applied by the same laser source using surgical pulse settings.

### CROSS-REFERENCE TO RELATED APPLICATION

This application claims priority to and benefit of U.S. provisional application Ser. No. 60/973,411, entitled “Methods and Apparatus for Laser Treatment of the Crystalline Lens” and filed on Sep. 18, 2007 by Ronald M. Kurtz, which is herein incorporated in its entirety by reference.

### BACKGROUND

This application relates to laser ophthalmic surgery.

Lens dysfunction occurs gradually over years as the lens continues to grow in size from birth through adulthood. See, e.g. “Adler's Physiology of the Eye: Clinical Application” authored by William Hart and published by Mosby-Year Book; Ninth Edition (August 1992). With progressive increase in size, the lens loses flexibility and becomes harder and harder. The inner and older lens fibers, constituting the nucleus, become increasingly removed from their nutritional source, the aqueous fluid. These processes result in two common lens pathologies, presbyopia and cataract.

Various surgical procedures have been proposed or used for surgery on the crystalline lens. Some of these procedures involve removal of the entire lens, leaving behind only the lens capsule. This removal can be performed using various techniques, including use of ultrasound, heated fluids or lasers. An artificial “intra ocular” lens of various materials and designs can be placed in the left-behind lens capsule. Some of these procedures provide lens surgery procedures that do not remove lens material and instead apply laser pulses at the lens to soften its hard nucleus, or to alter its shape. These methods attempt to attain these goals by inducing a biological response by the untreated eye tissue adjacent to the laser-treated target regions, see e.g. U.S. Pat. No. 6,322,556 to Gwon et al.

While offering the potential for beneficial outcomes, these techniques tend to fail to mitigate or correct the root cause for the lens dysfunction, may introduce the need for expensive prosthetics and raise the potential of significant complications.

### SUMMARY

Methods and apparatus are described for laser treatment of the crystalline lens. Implementations of the described methods and apparatus include a laser treatment of a lens of an eye, including defining a target boundary of a target region in the lens, applying surgical laser pulses to the target boundary effectively resulting in a separation of the target region from the rest of the lens, and removing the separated target region from the lens.

One implementation defines the target boundary from determining at least one of a transparency of a lens region, an optical density of a lens region, a refractive error of the lens irrespective of the source of the refractive error, a reduced accommodation of a lens region, an image of a lens region, a flexibility, elasticity or accommodation of a lens region, and individual or normative data of the lens.

The target boundary can be defined by generating probe bubbles in the lens and identifying a boundary separating two regions wherein a mechanical or optical characteristic of the probe bubbles is different in the two regions.

The target boundary can be defined by applying marker laser pulses to outline the target boundary. The marker laser pulses can be applied by a laser source using marker pulse settings and the surgical laser pulses can be applied by the same laser source using surgical pulse settings.

The target boundary can be defined by applying marker laser pulses to outline the target boundary and imaging the outlined target boundary in an iterative sequence.

The target boundary can be defined in a central region or in a peripheral region of the nucleus of the eye.

The target can be defined in a session separate from a session when the surgical laser pulses are applied or in the same session when the surgical laser pulses are applied.

The surgical pulses can be applied with a separation of generated surgical bubbles between 1 micron and 50 microns, a duration of the surgical laser pulses between 0.01 picoseconds and 50 picoseconds, an energy per surgical laser pulse between 0.5 μJ and 50 μJ, and a surgical laser pulse repetition rate between 10 kHz and 100 MHz.

The surgical pulses can be applied with settings between a lower threshold, identified based on the surgical laser pulses achieving a desired result and an upper threshold, identified based on the surgical laser pulses avoiding a damage to a selected tissue.

The target boundary can be defined and the surgical laser pulses can be applied before making an incision on the eye.

The separated target region can be removed by fragmenting the target region prior to the removal from the lens by photodisruption, using ultrasound, or heated fluids. In some implementations first surgical laser pulses can be applied to a posterior region of the target boundary, followed by applying fragmenting laser pulses to the target region, and finally surgical laser pulses can be applied to an anterior region of the target boundary.

The separated target region can be removed by forming an opening in the lens with photodisruption, an ultrasound-based method, a heated fluid-based method and a mechanical surgical method.

The separated target region can be aspirated through the formed opening.

The treatment may also include introducing a pharmacological agent, a medication, a fluid or an implantable device in a void left behind by the removed target.

In an implementation the physiologic functioning of a lens can be restored by identifying a volume of lens tissue to be removed, separating the identified volume of lens tissue from the surrounding lens tissue by laser-fragmenting a boundary of the identified volume of the lens tissue, removing the identified volume of lens tissue from the lens, and managing a reapproximation of a remaining lens portion to improve the functionality of the lens.

The lens tissue can be identified by comparing a size of the lens to normative data or to other ocular structures, determining a measure of lens flexibility or eye accommodation, determining a reduction of an optical transparency, and determining a refractive error.

The identified volume of lens tissue can be separated by outlining the boundary of the identified volume by marker laser pulses and separating the boundary of the identified volume by surgical laser pulses.

The identified volume of lens tissue can be removed by forming an opening in the lens and making corresponding incisions in a cornea of the eye.

The reapproximation of the remaining lens portion can be managed by infusing at least one of a medication and a fluid into a void left by the removal of the identified volume, and inserting an implantable device in the into a void left by the removal of the identified volume.

In some implementations, a laser surgical device for a treatment of a lens in an eye is provided to include an imaging module, configured to image the lens to provide information for defining a target boundary of a target region in the lens for treatment; a surgical laser module, configured to apply surgical laser pulses to the target boundary effectively resulting in a separation of the target region from the rest of the lens; and a surgical intervention module, configured to remove the separated target region from the lens. The imaging module may include an optical coherence tomography (OCT) imaging module.

These and other implementations are described in greater detail in the drawings, the description and the claims.

### BRIEF DESCRIPTION OF THE DRAWINGS

FIG. 1 illustrates an eye.

FIGS. 2A and 2B illustrate a target region in the lens of the eye.

FIG. 3 illustrates steps of a laser treatment.

FIGS. 4A-4C illustrate the steps of the laser treatment.

FIG. 5. illustrates the generation and use of probe bubbles.

FIGS. 6A and 6B illustrate the reapproximation of the lens after the removal of the target region.

FIG. 7 shows an example of an imaging-guided laser surgical system in which an imaging module is provided to provide imaging of a target to the laser control.

FIGS. 8-16 show examples of imaging-guided laser surgical systems with varying degrees of integration of a laser surgical system and an imaging system.

FIG. 17 shows an example of a method for performing laser surgery by suing an imaging-guided laser surgical system.

FIG. 18 shows an example of an image of an eye from an optical coherence tomography (OCT) imaging module.

FIGS. 19A, 19B, 19C and 19D show two examples of calibration samples for calibrating an imaging-guided laser surgical system.

FIG. 20 shows an example of attaching a calibration sample material to a patent interface in an imaging-guided laser surgical system for calibrating the system.

FIG. 21 shows an example of reference marks created by a surgical laser beam on a glass surface.

FIG. 22 shows an example of the calibration process and the post-calibration surgical operation for an imaging-guided laser surgical system.

FIGS. 23A and 23B show two operation modes of an exemplary imaging-guided laser surgical system that captures images of laser-induced photodisruption byproduct and the target issue to guide laser alignment.

FIGS. 24 and 25 show examples of laser alignment operations in imaging-guided laser surgical systems.

FIG. 26 shows an exemplary laser surgical system based on the laser alignment using the image of the photodisruption byproduct.

### DETAILED DESCRIPTION

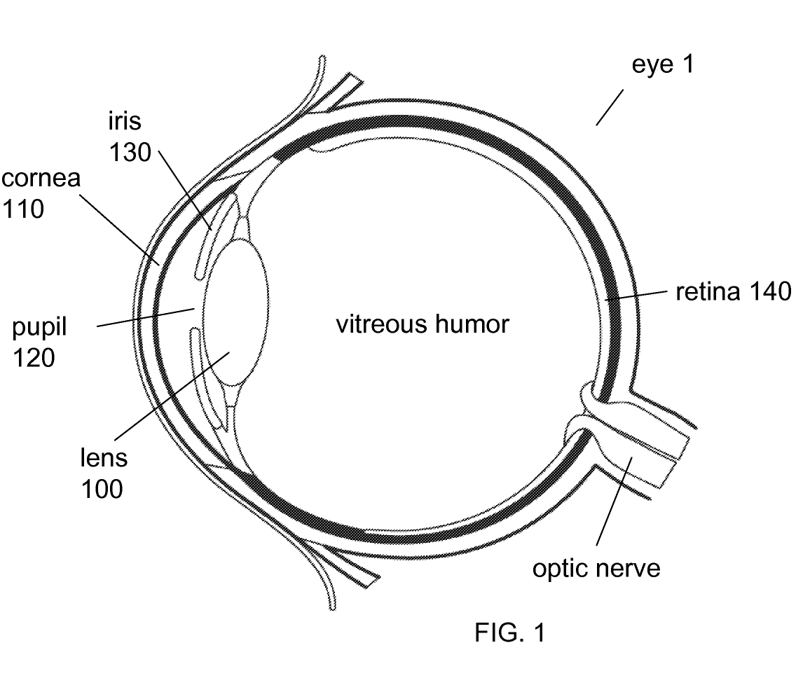

FIG. 1 illustrates the overall structure of the eye 1. The incident light propagates through the optical path which includes the cornea 110, the anterior chamber, the pupil 120, defined by the iris 130, the posterior chamber, the lens 100 and the vitreous humor. These optical elements guide the light on the retina 140.

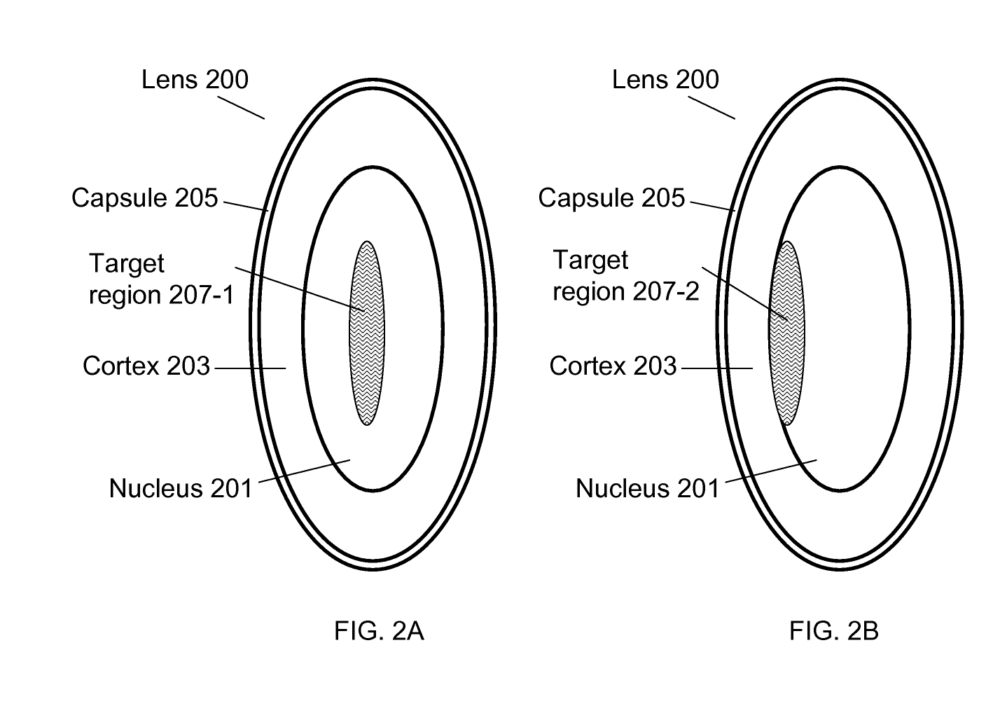

FIG. 2 illustrates a lens 200 in more detail. The lens 200 is sometimes referred to as crystalline lens because of the α, β, and γ crystalline proteins which make up about 90% of the lens. The crystalline lens has multiple optical functions in the eye, including its dynamic focusing capability. The lens is a unique tissue of the human body in that it continues to grow in size during gestation, after birth and throughout life. The lens grows by developing new lens fiber cells starting from the germinal center located on the equatorial periphery of the lens. The lens fibers are long, thin, transparent cells, with diameters typically between 4-7 microns and lengths of up to 12 mm. The oldest lens fibers are located centrally within the lens, forming the nucleus 201.

The nucleus 201 can be further subdivided into embryonic, fetal and adult nuclear zones. The new growth around the nucleus 201, referred to as cortex 203, develops in concentric ellipsoid layers, regions, or zones. Because the nucleus 201 and the cortex 203 are formed at different stages of the human development, their optical properties are distinct. While the lens increases in diameter over time, it may also undergo compaction so that the properties of the nucleus 201 and the surrounding cortex 203 may become even more different (Freel et al BMC Opthalmology 2003, vol. 3, p. 1).

As a result of this complex growth process, a typical lens 200 includes a harder nucleus 201 with an axial extent of about 2 mm, surrounded by a softer cortex 203 of axial width of 1-2 mm, contained by a much thinner capsule membrane 205, of typical width of about 20 microns. These values may change from person to person to a considerable degree.

FIG. 2 illustrates, lens fiber cells undergo progressive loss of cytoplasmic elements with the passage of time. Since no blood veins or lymphatics reach the lens to supply its inner zone, with advancing age the optical clarity, flexibility and other functional properties of the lens sometimes deteriorate. In some circumstances, including long-term ultraviolet exposure, exposure to radiation in general, denaturation of lens proteins, secondary effects of diseases such as diabetes, hypertension and advanced age, a region 207 of the nucleus 201 can become a region with undesirable properties.

As described above, many different undesirable properties can be developed by the lens, including being too hard to be properly shaped by the ciliary muscles, or having a reduced transparency. The results of such undesirable properties can be the development of presbyopia and cataract that increase in severity and incidence with age.

FIG. 2A illustrates that the target region 207-1 can be located in a central region in the nucleus 201.

FIG. 2B illustrates that the target region 207-2 can be located at a peripheral region of the nucleus 201.

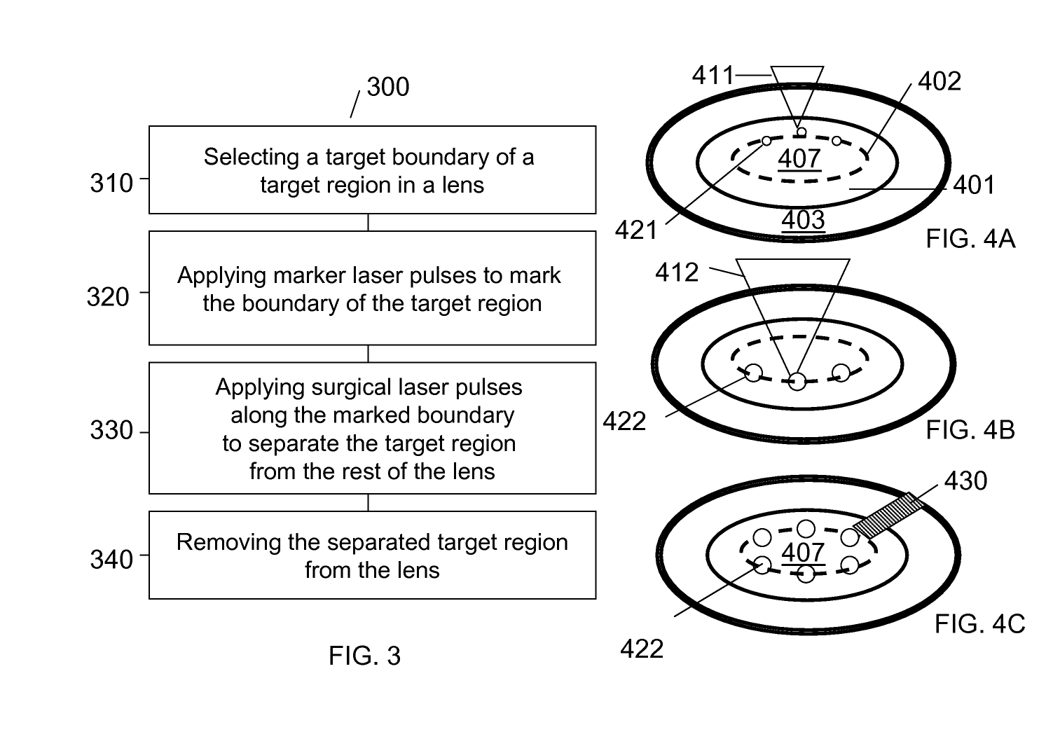

FIG. 3 illustrates that implementations of the present application improve the performance of the lens 200 by removing this target region 207.

FIGS. 4A-C illustrate certain implementations of such a laser treatment method, or surgical procedure, 300.

Step 310 may involve selecting a target region in a lens. The shape of the target region can be chosen based on several factors, which may include the following.

(1) The dimensions of the particular lens relative to desired lens dimensions, based on surveys of the population, or on experimental data of lens sizes either in isolation or relative to other ocular structures and/or function.

(2) Actual measurements of the global or regional transparency, optical density, elasticity, flexibility, or refractive power of the particular lens, when compared to desired values of these characteristics.

(3) Calculations and estimates that the lens tissue after the removal of the selected target region will relax to a desired and optically improved shape.

In one embodiment, an image of the lens is used to identify the target region 207. Such images can be created using a large number of known imaging techniques.

In some embodiments, the laser treatment method 300 involves applying laser-induced photodisruption. Laser-induced photodisruption or fragmentation includes ionizing a portion of the molecules in a targeted area. When parameters of the used laser pulses are above a “plasma threshold”, the laser pulses may generate an avalanche of secondary ionization processes. In many surgical procedures a large amount of energy is transferred to the targeted area in short bursts. These concentrated energy pulses may gasify the ionized region, leading to the formation of cavitation bubbles. These bubbles may form with a diameter of a few microns and expand with supersonic speeds to 50-100 microns. As the expansion of the bubbles decelerates to subsonic speeds, they may induce shockwaves in the surrounding tissue, causing secondary disruption. Both the bubbles themselves and the induced shockwaves disrupt the targeted tissue.

In recent years eye-surgical devices, often using excimer lasers, reached remarkable precision and control when applied to biological tissues. One of the limiting factors of the precision of these eye-surgical lasers is that the dynamics of the bubbles, generated by the lasers varies considerably depending on the targeted tissue. Bubbles can be controlled well in the harder nucleus 201 of the lens 200, because the generated bubbles remain largely confined, expanding only to a limited degree. In contrast, bubbles generated in the cortex 203 may expand to a considerable degree, and often in a hard-to-control manner.

For these reasons, implementations of step 310 sometimes select the target region 207 within the nucleus 201, where the bubbles, used for manipulating the target tissue can be well controlled. Other implementations may select a target region which includes portions outside the nucleus.

FIG. 4A illustrates an implementation of step 310, wherein a target region 407, defined by a target boundary 402 is selected within a nucleus 401.

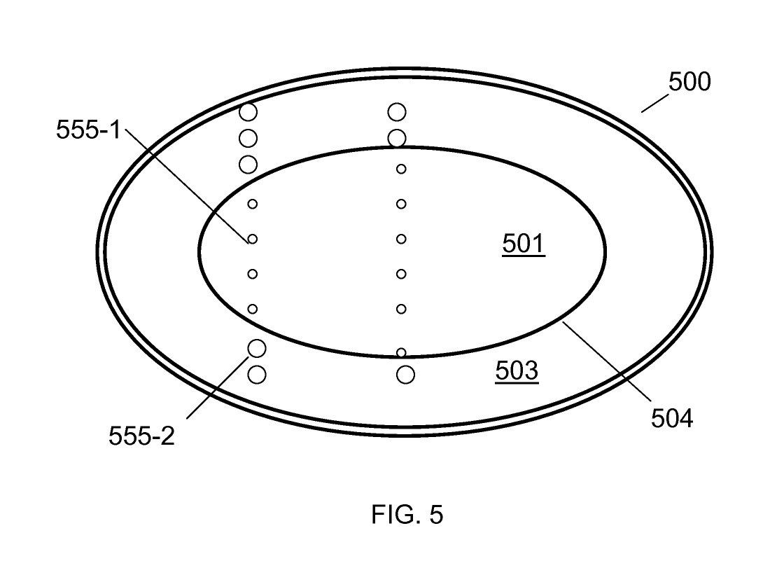

FIG. 5 illustrates a procedure to identify a nucleus 501 in a lens 500, based on generating bubbles in the lens 500 and observing their mechanical characteristics. A string of probe-bubbles 555 may be generated in the lens 500, for example, substantially parallel with a main axis of the eye, separated by a suitable distance, such as 10 to 100 microns. Other bubble strings can be generated in other areas of the lens. As illustrated, since the harder nucleus 501 shows more resistance against the expansion of the probe-bubbles 555, the probe-bubbles 555-1 inside the hard nucleus 501 may expand slower. In contrast, the cortex 503 may exert less resistance against the expansion of the bubbles and thus the probe-bubbles 555-2 outside the nucleus 501, in the cortex 503 may expand faster. A portion of the boundary 504 between the nucleus 501 and the cortex 503 can then be identified as the line or region which separates slow-expanding probe-bubbles 555-1 from fast-expanding probe-bubbles 555-2.

The expansion of the probe-bubbles 555 and the line separating the slow-expanding probe-bubbles 555-1 from the fast-expanding probe-bubbles 555-2 may be observed and tracked by an optical observation method. Many such methods are known, including various imaging techniques. Mapping out or otherwise recording these separation points or lines can be used to establish the boundary 504 between the softer lens regions and the hard lens region.

This implementation of step 310 can be pre-operative, i.e. performed prior to the surgical procedure 300, or intra-operative, i.e. performed as an early phase of the surgical procedure 300. When step 310 is performed in a pre-operative session, step 310 can be performed in a less regulated environment, such as outside of surgical settings.

Several other methods can be applied for step 310 as well. For example, optical or structural measurements can be performed prior to the surgical procedure 300 on the patient. Or, a database can be used, which correlates some other measurable characteristic of the eye to the size of the nucleus, e.g. using an age-dependent algorithm. In some cases an explicit calculation can be employed as well. In some cases data gained from cadaver studies can be utilized. It is also possible to generate the above bubble string, apply an ultrasound agitation, and then observe the induced oscillation of the bubbles, especially their frequency. From these observations, the hardness of the surrounding tissue can be inferred as well.

In some cases the method of Optical Coherence Tomography (OCT) can be utilized in step 310. Among other aspects, OCT can measure the opacity of the imaged tissue. From this measurement, the size of the bubbles and the hardness of the region can be inferred once again.

Once any of the above methods have been applied to determine the boundary of the nucleus 501, subsequent steps can be employed to select a target region within the nucleus 501. These steps can involve observing and analyzing the expanding probe-bubbles 555, or employing calculations or comparative database analysis based on the just-determined boundary of the nucleus. As described above in detail, the boundary of the target region can be selected by factoring in the desired changes of the lens, by calculating the target boundary necessary to improve the accommodation of the lens, or the transparency, among others.

Returning to FIG. 3, step 320 may involve applying marker laser pulses to mark the boundary of the selected target region. The marker laser pulses may generate marker bubbles outlining the boundary of the target region. These marker bubbles can be helpful to guide the placement and application of subsequent surgical laser pulses.

FIG. 4A illustrates that step 320 may involve applying marker laser pulses 411 to generate marker bubbles 412 outlining the identified target boundary 402 of the target region 407. In some implementations, the marker bubbles 412 are not meant to actually separate the target region from the rest of the nucleus. Therefore, the marker bubbles can be generated by applying only a lower or medium energy per pulse, and can be separated by larger distances, so as to only outline the boundary of the target.

In some implementations, generating the marker bubbles can be performed iteratively. A set of marker bubbles may be generated, followed by an imaging step to review the outlined region and compare it with the desired target region, and then generate a subsequent set of marker bubbles to approximate the desired target region more accurately.

Performing these steps iteratively and recording the settings of the laser apparatus which generated the marker bubbles at the desired location with the highest precision provides guidance for the operator of the laser apparatus to use the same or analogous settings when subsequently applying the more powerful surgical laser pulses.

In some implementations, steps 310 and 320 are part of defining the target boundary 402 of the target region 407.

In some implementations the boundary can be outlined by a non-laser plasma source.

FIG. 4B illustrates that step 330 may include applying surgical laser pulses 412 to the target boundary 402 marked by the marker bubbles 421. The surgical laser pulses 412 may generate surgical bubbles 422, which are capable of separating the target region 407 from the rest of the lens 400.

Laser parameters of the surgical laser 412 can be selected to generate surgical bubbles 422 sufficiently large that they substantially weaken the mechanical connection of the target region 407 to the rest of the lens 400, in effect perforating the lens tissue at the target boundary 402.

In some typical implementations, the energy per pulse of a surgical laser pulse 412 can be larger than that of a marker laser pulse 411, creating a surgical bubble 422 which is larger than a marker laser bubble 412.

Laser parameters for the surgical laser pulses may be selected as follows. In some cases the range of surgical bubble separation can be between 1 micron and 50 microns. The duration of the surgical laser pulses may vary in the range of 0.01 picoseconds to 50 picoseconds. In some patients particular results were achieved in the pulse duration range of 100 femtoseconds to 2 picoseconds. In some implementations, the laser energy per pulse can vary between 0.5 μJ and 50 μJ. The laser pulse repetition rate can vary between the thresholds of 10 kHz and 100 MHz.

These parameter ranges can be determined according to the effect of the laser pulses when applied with such parameters. The lower thresholds may be determined so that the laser pulses achieve the desired efficiency or effect. The upper thresholds may be determined so that the laser pulses do not cause harm to a tissue which is not to be damaged during the present surgical treatment, such as the retina.

Creating the surgical bubbles in an anterior portion of the target boundary 402 may perturb the optical pathways towards the deeper lying tissues of the lens, such as a posterior portion of the target boundary 402, since the surgical bubbles at the anterior boundary may scatter, reflect or absorb subsequent laser pulses. Therefore, in some implementations of step 330 the surgical bubbles can be first generated by surgical laser pulses focused at a posterior portion of the target boundary, followed by the generation of surgical bubbles at an anterior portion of the boundary.

In some implementations the steps 310, 320 and 330 are carried out without opening the eye with incisions. In such procedures the optical pathways are not disturbed and the focusing and controlling of the laser pulses can be performed with high precision.

Moreover, methods without incisions can be practiced without fluid management. In open-eye surgeries which employ incisions the fluids of the eye, such as the aqueous humor of the antechamber, start seeping out through the incision. Since these fluids play a vital role in propping up the structure of the eye as well as in maintaining a clear optical pathway, the fluids need to be replenished. In such procedures often a computer controls the administering of a viscoelastic to replace the lost eye-fluids. The present method 300 and its equivalents do not require such fluid management during steps 310-330, thus providing superior control and ease of implementation.

In some implementations the steps 310, 320 and 330 are carried out in an integrated manner, by utilizing the same laser device. In step 310, the probe bubbles may be generated with a low energy-per-pulse setting, in step 320 the marker bubbles may be generated by with a medium energy-per-pulse setting, and finally in step 330 the surgical bubbles can be generated with a high energy-per-pulse setting, all of them utilizing the same laser device.

Eye-surgical procedures often have to be carried out under tight time-limitations. Surgeries rarely last longer than two minutes, and often have to be as short as one minute. It has qualitative advantages that several steps of the present integrated surgical method can be carried out by a single laser device by changing only its settings instead of changing the device itself. These advantages include allowing the surgeon to use the limited surgical time for other procedures which otherwise would have been prohibited by the strict time limits, or to perform the same procedures with greater precision.

Step 340 may involve removing the target region 407, which has been separated from the rest of the lens 400 by surgical bubbles 422, through a suitable opening 430.

In the implementation of FIG. 2A the target region can be a central portion of the nucleus, or it can be essentially the entire nucleus.

In the implementation of FIG. 2B, when the target region is a peripheral portion of the nucleus, this procedure is sometimes referred to as “sculpting” the lens.

While surgical bubbles may have weakened the connection of the target region 407 to the rest of the lens 400 to a considerable degree, this removal step 340 may involve a small amount of mechanical separation, such as a limited amount of tearing. Nevertheless, the amount of tearing in this process is qualitatively less than in ultrasound-based procedures, and thus does not lead to substantial unwanted effects, which would require follow up intervention.

The target region 407 can be removed in different ways. Some methods include removing the separated target region 407 as a whole. Other methods involve disrupting the target region into smaller fragments. This fragmentation of the target region 407 can be achieved by applying laser pulses, ultrasound, heated fluids, or mechanical means. The size of the fragments may vary in a wide range: in some cases the fragments can be so small that the target region in effect is emulsified and can be removed by aspiration. In this case an aspiration probe can be attached to, or inserted into the lens 400 at the opening 430.

Creating the surgical bubbles of step 330 in an anterior portion of the target boundary 402 may perturb the optical pathways towards the deeper lying tissues of the lens, such as to the target region 407 itself when the fragmentation of the target region is performed as part of step 340, since the surgical bubbles at the anterior boundary may scatter, reflect or absorb the subsequent laser pulses. Therefore, in some implementations step 330 and 340 can be carried out in an integrated or combined manner. Some implementations may start with applying surgical laser pulses to a posterior portion of the target boundary as part of step 330, continue with applying fragmenting laser pulses to the target region to fragment at least a portion of the target region 407 as part of step 340, and finish with applying surgical laser pulses to an anterior portion of the boundary as part of step 330.

In some implementations a peripheral boundary may be established with either the posterior or the anterior target boundary depending on the geometry required to access the target region.

The opening 430 can be formed by a large number of ways. These include applying a pulsed laser beam and cause photodisruption to an elongated portion of the lens 400, so formed that the separated target region 407 can be removed through it. Other methods, based on ultrasound or surgical interventions can also be used.

The suitable opening 430 can be formed off the main axis, or the center, of the lens 400, as shown in FIG. 4C. Such a choice limits the impact and possible distortion of the optical path of the treated eye caused by the formation of the opening 430. An angle of the opening 430 can vary in a wide range, from being nearly parallel to the main axis of the lens to be lying near the equatorial plane of the lens.

In implementations when the target region 407 is larger and thus a diameter of the opening 430 is larger as well, the opening 430 may be formed centrally, so that the contours of the opening 430 impact the main optical pathways only to a limited degree.

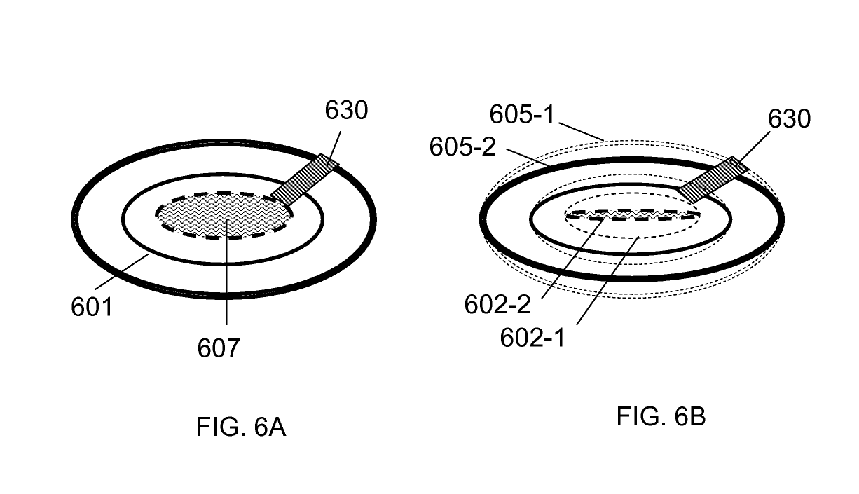

FIGS. 6A-B illustrate the reaction of a lens 600 to the removal of the target region 607, located in nucleus 601. FIG. 6A illustrates a target region 607 before the removal through a suitable opening 630.

FIG. 6B illustrates that after the target region 607 has been removed, a void is left behind in the nucleus 601, which can be essentially empty or contain some level of debris or fluids.

After the target region 607 is removed, the remaining portion of the lens 600 assumes a post-treatment shape that may have improved accommodation, refraction and/or clarity. For example, if a portion of the hard nucleus 601 has been removed, the ciliary muscles may control the shape of the remaining lens 600 with less effort, thus improving the accommodation of the lens.

The change of shape of the lens 600 is sometimes referred to as “reapproximation”. The shape the target region 607 in step 310 can be selected to enhance and optimize the desired improvement of the lens when the remaining lens tissue reapproximates. The removal of the target regions 607 is sometimes referred to as “debulking” the lens as well.

This reapproximation is illustrated in FIG. 6B: before the removal of the target region 607 the lens capsule has the shape 605-1 and the boundary of the target region has the shape 602-1. After the removal of the target region 607 the lens capsule reapproximates to a shape 605-2 and the boundary of the target region to the much-reduced 602-2.

In the illustrated implementation of method 300 a curvature of the lens 600 changed as a result of the eye-treatment. Also, since the removed target portion 607 belonged to the harder nucleus, the accommodation of the reapproximated lens improved as well.

These improvements enable the remaining reapproximated lens to provide improved physiologic and optical functioning, thus possibly eliminating the need of prosthetic devices.

In some implementations pharmacologic agents, any kind of implantable devices or their combination may be placed in the void left behind by the removal of the target region 607. Placing these into the void can augment a function of the eye, aid or hinder tissue reactions, or otherwise improve surgical outcomes.

The shape of the target region 607 can be controlled to provide correction of refractive errors, irrespective of their source. These refractive errors may emerge from the cornea or an overall shape of the eye. A lens, debulked by the above procedure, can provide better functioning of the remaining lens tissue optically and mechanically, thus being useful for the treatment of presbyopia or cataract.

In contrast to some other surgical procedures, the spatial extent of the target region in the presently described laser treatment 300 can be controlled with high precision. Therefore, the effects of the laser treatment 300 can be considerably more significant and reproducible than the process described by Gwon et al, which relies on a hard-to-control biological response of the tissue adjacent to the treated regions.

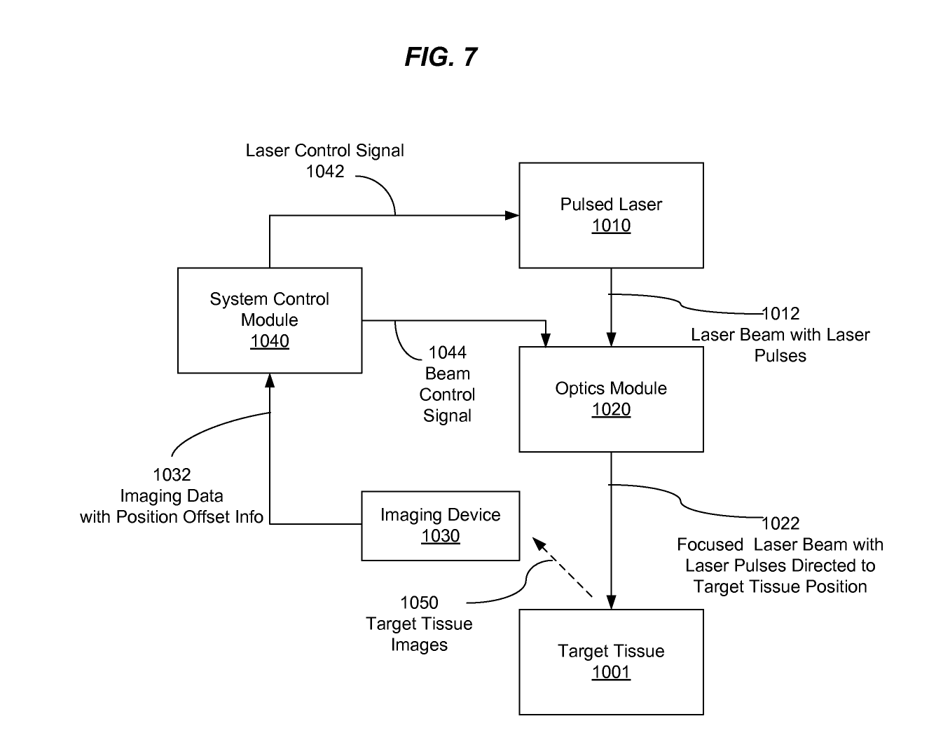

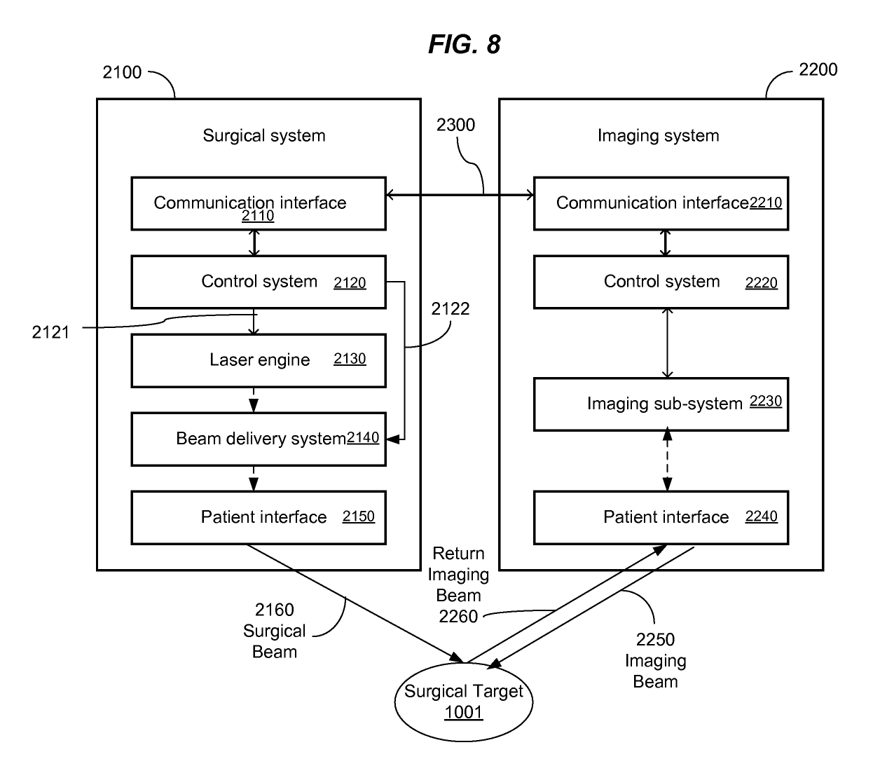

FIGS. 7-26 illustrate embodiments of a laser surgery system in relation to the above photodisruptive laser treatment.

One important aspect of laser surgical procedures is precise control and aiming of a laser beam, e.g., the beam position and beam focusing. Laser surgery systems can be designed to include laser control and aiming tools to precisely target laser pulses to a particular target inside the tissue. In various nanosecond photodisruptive laser surgical systems, such as the Nd:YAG laser systems, the required level of targeting precision is relatively low. This is in part because the laser energy used is relatively high and thus the affected tissue area is also relatively large, often covering an impacted area with a dimension in the hundreds of microns. The time between laser pulses in such systems tend to be long and manual controlled targeting is feasible and is commonly used. One example of such manual targeting mechanisms is a biomicroscope to visualize the target tissue in combination with a secondary laser source used as an aiming beam. The surgeon manually moves the focus of a laser focusing lens, usually with a joystick control, which is parfocal (with or without an offset) with their image through the microscope, so that the surgical beam or aiming beam is in best focus on the intended target.

Such techniques designed for use with low repetition rate laser surgical systems may be difficult to use with high repetition rate lasers operating at thousands of shots per second and relatively low energy per pulse. In surgical operations with high repetition rate lasers, much higher precision may be required due to the small effects of each single laser pulse and much higher positioning speed may be required due to the need to deliver thousands of pulses to new treatment areas very quickly.

Examples of high repetition rate pulsed lasers for laser surgical systems include pulsed lasers at a pulse repetition rate of thousands of shots per second or higher with relatively low energy per pulse. Such lasers use relatively low energy per pulse to localize the tissue effect caused by laser-induced photodisruption, e.g., the impacted tissue area by photodisruption on the order of microns or tens of microns. This localized tissue effect can improve the precision of the laser surgery and can be desirable in certain surgical procedures such as laser eye surgery. In one example of such surgery, placement of many hundred, thousands or millions of contiguous, nearly contiguous or pulses separated by known distances, can be used to achieve certain desired surgical effects, such as tissue incisions, separations or fragmentation.

Various surgical procedures using high repetition rate photodisruptive laser surgical systems with shorter laser pulse durations may require high precision in positioning each pulse in the target tissue under surgery both in an absolute position with respect to a target location on the target tissue and a relative position with respect to preceding pulses. For example, in some cases, laser pulses may be required to be delivered next to each other with an accuracy of a few microns within the time between pulses, which can be on the order of microseconds. Because the time between two sequential pulses is short and the precision requirement for the pulse alignment is high, manual targeting as used in low repetition rate pulsed laser systems may be no longer adequate or feasible.

One technique to facilitate and control precise, high speed positioning requirement for delivery of laser pulses into the tissue is attaching a applanation plate made of a transparent material such as a glass with a predefined contact surface to the tissue so that the contact surface of the applanation plate forms a well-defined optical interface with the tissue. This well-defined interface can facilitate transmission and focusing of laser light into the tissue to control or reduce optical aberrations or variations (such as due to specific eye optical properties or changes that occur with surface drying) that are most critical at the air-tissue interface, which in the eye is at the anterior surface of the cornea. Contact lenses can be designed for various applications and targets inside the eye and other tissues, including ones that are disposable or reusable. The contact glass or applanation plate on the surface of the target tissue can be used as a reference plate relative to which laser pulses are focused through the adjustment of focusing elements within the laser delivery system. This use of a contact glass or applanation plate provides better control of the optical qualities of the tissue surface and thus allow laser pulses to be accurately placed at a high speed at a desired location (interaction point) in the target tissue relative to the applanation reference plate with little optical distortion of the laser pulses.

One way for implementing an applanation plate on an eye is to use the applanation plate to provide a positional reference for delivering the laser pulses into a target tissue in the eye. This use of the applanation plate as a positional reference can be based on the known desired location of laser pulse focus in the target with sufficient accuracy prior to firing the laser pulses and that the relative positions of the reference plate and the individual internal tissue target must remain constant during laser firing. In addition, this method can require the focusing of the laser pulse to the desired location to be predictable and repeatable between eyes or in different regions within the same eye. In practical systems, it can be difficult to use the applanation plate as a positional reference to precisely localize laser pulses intraocularly because the above conditions may not be met in practical systems.

For example, if the crystalline lens is the surgical target, the precise distance from the reference plate on the surface of the eye to the target tends to vary due to the presence of collapsible structures, such as the cornea itself, the anterior chamber, and the iris. Not only is their considerable variability in the distance between the applanated cornea and the lens between individual eyes, but there can also be variation within the same eye depending on the specific surgical and applanation technique used by the surgeon. In addition, there can be movement of the targeted lens tissue relative to the applanated surface during the firing of the thousands of laser pulses required for achieving the surgical effect, further complicating the accurate delivery of pulses. In addition, structure within the eye may move due to the build-up of photodisruptive byproducts, such as cavitation bubbles. For example, laser pulses delivered to the crystalline lens can cause the lens capsule to bulge forward, requiring adjustment to target this tissue for subsequent placement of laser pulses. Furthermore, it can be difficult to use computer models and simulations to predict, with sufficient accuracy, the actual location of target tissues after the applanation plate is removed and to adjust placement of laser pulses to achieve the desired localization without applanation in part because of the highly variable nature of applanation effects, which can depend on factors particular to the individual cornea or eye, and the specific surgical and applanation technique used by a surgeon.

In addition to the physical effects of applanation that disproportionably affect the localization of internal tissue structures, in some surgical processes, it may be desirable for a targeting system to anticipate or account for nonlinear characteristics of photodisruption which can occur when using short pulse duration lasers. Photodisruption is a nonlinear optical process in the tissue material and can cause complications in beam alignment and beam targeting. For example, one of the nonlinear optical effects in the tissue material when interacting with laser pulses during the photodisruption is that the refractive index of the tissue material experienced by the laser pulses is no longer a constant but varies with the intensity of the light. Because the intensity of the light in the laser pulses varies spatially within the pulsed laser beam, along and across the propagation direction of the pulsed laser beam, the refractive index of the tissue material also varies spatially. One consequence of this nonlinear refractive index is self-focusing or self-defocusing in the tissue material that changes the actual focus of and shifts the position of the focus of the pulsed laser beam inside the tissue. Therefore, a precise alignment of the pulsed laser beam to each target tissue position in the target tissue may also need to account for the nonlinear optical effects of the tissue material on the laser beam. In addition, it may be necessary to adjust the energy in each pulse to deliver the same physical effect in different regions of the target due to different physical characteristics, such as hardness, or due to optical considerations such as absorption or scattering of laser pulse light traveling to a particular region. In such cases, the differences in non-linear focusing effects between pulses of different energy values can also affect the laser alignment and laser targeting of the surgical pulses.

Thus, in surgical procedures in which non superficial structures are targeted, the use of a superficial applanation plate based on a positional reference provided by the applanation plate may be insufficient to achieve precise laser pulse localization in internal tissue targets. The use of the applanation plate as the reference for guiding laser delivery may require measurements of the thickness and plate position of the applanation plate with high accuracy because the deviation from nominal is directly translated into a depth precision error. High precision applanation lenses can be costly, especially for single use disposable applanation plates.

The techniques, apparatus and systems described in this document can be implemented in ways that provide a targeting mechanism to deliver short laser pulses through an applanation plate to a desired localization inside the eye with precision and at a high speed without requiring the known desired location of laser pulse focus in the target with sufficient accuracy prior to firing the laser pulses and without requiring that the relative positions of the reference plate and the individual internal tissue target remain constant during laser firing. As such, the present techniques, apparatus and systems can be used for various surgical procedures where physical conditions of the target tissue under surgery tend to vary and are difficult to control and the dimension of the applanation lens tends to vary from one lens to another. The present techniques, apparatus and systems may also be used for other surgical targets where distortion or movement of the surgical target relative to the surface of the structure is present or non-linear optical effects make precise targeting problematic. Examples for such surgical targets different from the eye include the heart, deeper tissue in the skin and others.

The present techniques, apparatus and systems can be implemented in ways that maintain the benefits provided by an applanation plate, including, for example, control of the surface shape and hydration, as well as reductions in optical distortion, while providing for the precise localization of photodisruption to internal structures of the applanated surface. This can be accomplished through the use of an integrated imaging device to localize the target tissue relative to the focusing optics of the delivery system. The exact type of imaging device and method can vary and may depend on the specific nature of the target and the required level of precision.

An applanation lens may be implemented with another mechanism to fix the eye to prevent translational and rotational movement of the eye. Examples of such fixation devices include the use of a suction ring. Such fixation mechanism can also lead to unwanted distortion or movement of the surgical target. The present techniques, apparatus and systems can be implemented to provide, for high repetition rate laser surgical systems that utilize an applanation plate and/or fixation means for non-superficial surgical targets, a targeting mechanism to provide intraoperative imaging to monitor such distortion and movement of the surgical target.

Specific examples of laser surgical techniques, apparatus and systems are described below to use an optical imaging module to capture images of a target tissue to obtain positioning information of the target tissue, e.g., before and during a surgical procedure. Such obtained positioning information can be used to control the positioning and focusing of the surgical laser beam in the target tissue to provide accurate control of the placement of the surgical laser pulses in high repetition rate laser systems. In one implementation, during a surgical procedure, the images obtained by the optical imaging module can be used to dynamically control the position and focus of the surgical laser beam. In addition, lower energy and shot laser pulses tend to be sensitive to optical distortions, such a laser surgical system can implement an applanation plate with a flat or curved interface attaching to the target tissue to provide a controlled and stable optical interface between the target tissue and the surgical laser system and to mitigate and control optical aberrations at the tissue surface.

As an example, FIG. 7 shows a laser surgical system based on optical imaging and applanation. This system includes a pulsed laser 1010 to produce a surgical laser beam 1012 of laser pulses, and an optics module 1020 to receive the surgical laser beam 1012 and to focus and direct the focused surgical laser beam 1022 onto a target tissue 1001, such as an eye, to cause photodisruption in the target tissue 1001. An applanation plate can be provided to be in contact with the target tissue 1001 to produce an interface for transmitting laser pulses to the target tissue 1001 and light coming from the target tissue 1001 through the interface. Notably, an optical imaging device 1030 is provided to capture light 1050 carrying target tissue images 1050 or imaging information from the target tissue 1001 to create an image of the target tissue 1001. The imaging signal 1032 from the imaging device 1030 is sent to a system control module 1040. The system control module 1040 operates to process the captured images from the image device 1030 and to control the optics module 1020 to adjust the position and focus of the surgical laser beam 1022 at the target tissue 101 based on information from the captured images. The optics module 120 can include one or more lenses and may further include one or more reflectors. A control actuator can be included in the optics module 1020 to adjust the focusing and the beam direction in response to a beam control signal 1044 from the system control module 1040. The control module 1040 can also control the pulsed laser 1010 via a laser control signal 1042.

The optical imaging device 1030 may be implemented to produce an optical imaging beam that is separate from the surgical laser beam 1022 to probe the target tissue 1001 and the returned light of the optical imaging beam is captured by the optical imaging device 1030 to obtain the images of the target tissue 1001. One example of such an optical imaging device 1030 is an optical coherence tomography (OCT) imaging module which uses two imaging beams, one probe beam directed to the target tissue 1001 thought the applanation plate and another reference beam in a reference optical path, to optically interfere with each other to obtain images of the target tissue 1001. In other implementations, the optical imaging device 1030 can use scattered or reflected light from the target tissue 1001 to capture images without sending a designated optical imaging beam to the target tissue 1001. For example, the imaging device 1030 can be a sensing array of sensing elements such as CCD or CMS sensors. For example, the images of photodisruption byproduct produced by the surgical laser beam 1022 may be captured by the optical imaging device 1030 for controlling the focusing and positioning of the surgical laser beam 1022. When the optical imaging device 1030 is designed to guide surgical laser beam alignment using the image of the photodisruption byproduct, the optical imaging device 1030 captures images of the photodisruption byproduct such as the laser-induced bubbles or cavities. The imaging device 1030 may also be an ultrasound imaging device to capture images based on acoustic images.

The system control module 1040 processes image data from the imaging device 1030 that includes the position offset information for the photodisruption byproduct from the target tissue position in the target tissue 1001. Based on the information obtained from the image, the beam control signal 1044 is generated to control the optics module 1020 which adjusts the laser beam 1022. A digital processing unit can be included in the system control module 1040 to perform various data processing for the laser alignment.

The above techniques and systems can be used deliver high repetition rate laser pulses to subsurface targets with a precision required for contiguous pulse placement, as needed for cutting or volume disruption applications. This can be accomplished with or without the use of a reference source on the surface of the target and can take into account movement of the target following applanation or during placement of laser pulses.

The applanation plate in the present systems is provided to facilitate and control precise, high speed positioning requirement for delivery of laser pulses into the tissue. Such an applanation plate can be made of a transparent material such as a glass with a predefined contact surface to the tissue so that the contact surface of the applanation plate forms a well-defined optical interface with the tissue. This well-defined interface can facilitate transmission and focusing of laser light into the tissue to control or reduce optical aberrations or variations (such as due to specific eye optical properties or changes that occur with surface drying) that are most critical at the air-tissue interface, which in the eye is at the anterior surface of the cornea. A number of contact lenses have been designed for various applications and targets inside the eye and other tissues, including ones that are disposable or reusable. The contact glass or applanation plate on the surface of the target tissue is used as a reference plate relative to which laser pulses are focused through the adjustment of focusing elements within the laser delivery system relative. Inherent in such an approach are the additional benefits afforded by the contact glass or applanation plate described previously, including control of the optical qualities of the tissue surface. Accordingly, laser pulses can be accurately placed at a high speed at a desired location (interaction point) in the target tissue relative to the applanation reference plate with little optical distortion of the laser pulses.

The optical imaging device 1030 in FIG. 7 captures images of the target tissue 1001 via the applanation plate. The control module 1040 processes the captured images to extract position information from the captured images and uses the extracted position information as a position reference or guide to control the position and focus of the surgical laser beam 1022. This imaging-guided laser surgery can be implemented without relying on the applanation plate as a position reference because the position of the applanation plate tends to change due to various factors as discussed above. Hence, although the applanation plate provides a desired optical interface for the surgical laser beam to enter the target tissue and to capture images of the target tissue, it may be difficult to use the applanation plate as a position reference to align and control the position and focus of the surgical laser beam for accurate delivery of laser pulses. The imaging-guided control of the position and focus of the surgical laser beam based on the imaging device 1030 and the control module 1040 allows the images of the target tissue 1001, e.g., images of inner structures of an eye, to be used as position references, without using the applanation plate to provide a position reference.

In addition to the physical effects of applanation that disproportionably affect the localization of internal tissue structures, in some surgical processes, it may be desirable for a targeting system to anticipate or account for nonlinear characteristics of photodisruption which can occur when using short pulse duration lasers. Photodisruption can cause complications in beam alignment and beam targeting. For example, one of the nonlinear optical effects in the tissue material when interacting with laser pulses during the photodisruption is that the refractive index of the tissue material experienced by the laser pulses is no longer a constant but varies with the intensity of the light. Because the intensity of the light in the laser pulses varies spatially within the pulsed laser beam, along and across the propagation direction of the pulsed laser beam, the refractive index of the tissue material also varies spatially. One consequence of this nonlinear refractive index is self-focusing or self-defocusing in the tissue material that changes the actual focus of and shifts the position of the focus of the pulsed laser beam inside the tissue. Therefore, a precise alignment of the pulsed laser beam to each target tissue position in the target tissue may also need to account for the nonlinear optical effects of the tissue material on the laser beam. The energy of the laser pulses may be adjusted to deliver the same physical effect in different regions of the target due to different physical characteristics, such as hardness, or due to optical considerations such as absorption or scattering of laser pulse light traveling to a particular region. In such cases, the differences in non-linear focusing effects between pulses of different energy values can also affect the laser alignment and laser targeting of the surgical pulses. In this regard, the direct images obtained from the target issue by the imaging device 1030 can be used to monitor the actual position of the surgical laser beam 1022 which reflects the combined effects of nonlinear optical effects in the target tissue and provide position references for control of the beam position and beam focus.

The techniques, apparatus and systems described here can be used in combination of an applanation plate to provide control of the surface shape and hydration, to reduce optical distortion, and provide for precise localization of photodisruption to internal structures through the applanated surface. The imaging-guided control of the beam position and focus described here can be applied to surgical systems and procedures that use means other than applanation plates to fix the eye, including the use of a suction ring which can lead to distortion or movement of the surgical target.

The following sections first describe examples of techniques, apparatus and systems for automated imaging-guided laser surgery based on varying degrees of integration of imaging functions into the laser control part of the systems. An optical or other modality imaging module, such as an OCT imaging module, can be used to direct a probe light or other type of beam to capture images of a target tissue, e.g., structures inside an eye. A surgical laser beam of laser pulses such as femtosecond or picosecond laser pulses can be guided by position information in the captured images to control the focusing and positioning of the surgical laser beam during the surgery. Both the surgical laser beam and the probe light beam can be sequentially or simultaneously directed to the target tissue during the surgery so that the surgical laser beam can be controlled based on the captured images to ensure precision and accuracy of the surgery.

Such imaging-guided laser surgery can be used to provide accurate and precise focusing and positioning of the surgical laser beam during the surgery because the beam control is based on images of the target tissue following applanation or fixation of the target tissue, either just before or nearly simultaneously with delivery of the surgical pulses. Notably, certain parameters of the target tissue such as the eye measured before the surgery may change during the surgery due to various factor such as preparation of the target tissue (e.g., fixating the eye to an applanation lens) and the alternation of the target tissue by the surgical operations. Therefore, measured parameters of the target tissue prior to such factors and/or the surgery may no longer reflect the physical conditions of the target tissue during the surgery. The present imaging-guided laser surgery can mitigate technical issues in connection with such changes for focusing and positioning the surgical laser beam before and during the surgery.

The present imaging-guided laser surgery may be effectively used for accurate surgical operations inside a target tissue. For example, when performing laser surgery inside the eye, laser light is focused inside the eye to achieve optical breakdown of the targeted tissue and such optical interactions can change the internal structure of the eye. For example, the crystalline lens can change its position, shape, thickness and diameter during accommodation, not only between prior measurement and surgery but also during surgery. Attaching the eye to the surgical instrument by mechanical means can change the shape of the eye in a not well defined way and further, the change can vary during surgery due to various factors, e.g., patient movement. Attaching means include fixating the eye with a suction ring and applanating the eye with a flat or curved lens. These changes amount to as much as a few millimeters. Mechanically referencing and fixating the surface of the eye such as the anterior surface of the cornea or limbus does not work well when performing precision laser microsurgery inside the eye.

The post preparation or near simultaneous imaging in the present imaging-guided laser surgery can be used to establish three-dimensional positional references between the inside features of the eye and the surgical instrument in an environment where changes occur prior to and during surgery. The positional reference information provided by the imaging prior to applanation and/or fixation of the eye, or during the actual surgery reflects the effects of changes in the eye and thus provides an accurate guidance to focusing and positioning of the surgical laser beam. A system based on the present imaging-guided laser surgery can be configured to be simple in structure and cost efficient. For example, a portion of the optical components associated with guiding the surgical laser beam can be shared with optical components for guiding the probe light beam for imaging the target tissue to simplify the device structure and the optical alignment and calibration of the imaging and surgical light beams.

The imaging-guided laser surgical systems described below use the OCT imaging as an example of an imaging instrument and other non-OCT imaging devices may also be used to capture images for controlling the surgical lasers during the surgery. As illustrated in the examples below, integration of the imaging and surgical subsystems can be implemented to various degrees. In the simplest form without integrating hardware, the imaging and laser surgical subsystems are separated and can communicate to one another through interfaces. Such designs can provide flexibility in the designs of the two subsystems. Integration between the two subsystems, by some hardware components such as a patient interface, further expands the functionality by offering better registration of surgical area to the hardware components, more accurate calibration and may improve workflow. As the degree of integration between the two subsystems increases, such a system may be made increasingly cost-efficient and compact and system calibration will be further simplified and more stable over time. Examples for imaging-guided laser systems in FIGS. 8-16 are integrated at various degrees of integration.

One implementation of a present imaging-guided laser surgical system, for example, includes a surgical laser that produces a surgical laser beam of surgical laser pulses that cause surgical changes in a target tissue under surgery; a patient interface mount that engages a patient interface in contact with the target tissue to hold the target tissue in position; and a laser beam delivery module located between the surgical laser and the patient interface and configured to direct the surgical laser beam to the target tissue through the patient interface. This laser beam delivery module is operable to scan the surgical laser beam in the target tissue along a predetermined surgical pattern. This system also includes a laser control module that controls operation of the surgical laser and controls the laser beam delivery module to produce the predetermined surgical pattern and an OCT module positioned relative to the patient interface to have a known spatial relation with respect to the patient interface and the target issue fixed to the patient interface. The OCT module is configured to direct an optical probe beam to the target tissue and receive returned probe light of the optical probe beam from the target tissue to capture OCT images of the target tissue while the surgical laser beam is being directed to the target tissue to perform an surgical operation so that the optical probe beam and the surgical laser beam are simultaneously present in the target tissue. The OCT module is in communication with the laser control module to send information of the captured OCT images to the laser control module.

In addition, the laser control module in this particular system responds to the information of the captured OCT images to operate the laser beam delivery module in focusing and scanning of the surgical laser beam and adjusts the focusing and scanning of the surgical laser beam in the target tissue based on positioning information in the captured OCT images.

In some implementations, acquiring a complete image of a target tissue may not be necessary for registering the target to the surgical instrument and it may be sufficient to acquire a portion of the target tissue, e.g., a few points from the surgical region such as natural or artificial landmarks. For example, a rigid body has six degrees of freedom in 3D space and six independent points would be sufficient to define the rigid body. When the exact size of the surgical region is not known, additional points are needed to provide the positional reference. In this regard, several points can be used to determine the position and the curvature of the anterior and posterior surfaces, which are normally different, and the thickness and diameter of the crystalline lens of the human eye. Based on these data a body made up from two halves of ellipsoid bodies with given parameters can approximate and visualize a crystalline lens for practical purposes. In another implementation, information from the captured image may be combined with information from other sources, such as pre-operative measurements of lens thickness that are used as an input for the controller.

FIG. 8 shows one example of an imaging-guided laser surgical system with separated laser surgical system 2100 and imaging system 2200. The laser surgical system 2100 includes a laser engine 2130 with a surgical laser that produces a surgical laser beam 2160 of surgical laser pulses. A laser beam delivery module 2140 is provided to direct the surgical laser beam 2160 from the laser engine 2130 to the target tissue 1001 through a patient interface 2150 and is operable to scan the surgical laser beam 2160 in the target tissue 1001 along a predetermined surgical pattern. A laser control module 2120 is provided to control the operation of the surgical laser in the laser engine 2130 via a communication channel 2121 and controls the laser beam delivery module 2140 via a communication channel 2122 to produce the predetermined surgical pattern. A patient interface mount is provided to engage the patient interface 2150 in contact with the target tissue 1001 to hold the target tissue 1001 in position. The patient interface 2150 can be implemented to include a contact lens or applanation lens with a flat or curved surface to conformingly engage to the anterior surface of the eye and to hold the eye in position.

The imaging system 2200 in FIG. 8 can be an OCT module positioned relative to the patient interface 2150 of the surgical system 2100 to have a known spatial relation with respect to the patient interface 2150 and the target issue 1001 fixed to the patient interface 2150. This OCT module 2200 can be configured to have its own patient interface 2240 for interacting with the target tissue 1001. The imaging system 2200 includes an imaging control module 2220 and an imaging sub-system 2230. The sub-system 2230 includes a light source for generating imaging beam 2250 for imaging the target 1001 and an imaging beam delivery module to direct the optical probe beam or imaging beam 2250 to the target tissue 1001 and receive returned probe light 2260 of the optical imaging beam 2250 from the target tissue 1001 to capture OCT images of the target tissue 1001. Both the optical imaging beam 2250 and the surgical beam 2160 can be simultaneously directed to the target tissue 1001 to allow for sequential or simultaneous imaging and surgical operation.

As illustrated in FIG. 8, communication interfaces 2110 and 2210 are provided in both the laser surgical system 2100 and the imaging system 2200 to facilitate the communications between the laser control by the laser control module 2120 and imaging by the imaging system 2200 so that the OCT module 2200 can send information of the captured OCT images to the laser control module 2120. The laser control module 2120 in this system responds to the information of the captured OCT images to operate the laser beam delivery module 2140 in focusing and scanning of the surgical laser beam 2160 and dynamically adjusts the focusing and scanning of the surgical laser beam 2160 in the target tissue 1001 based on positioning information in the captured OCT images. The integration between the laser surgical system 2100 and the imaging system 2200 is mainly through communication between the communication interfaces 2110 and 2210 at the software level.

In this and other examples, various subsystems or devices may also be integrated. For example, certain diagnostic instruments such as wavefront aberrometers, corneal topography measuring devices may be provided in the system, or pre-operative information from these devices can be utilized to augment intra-operative imaging.

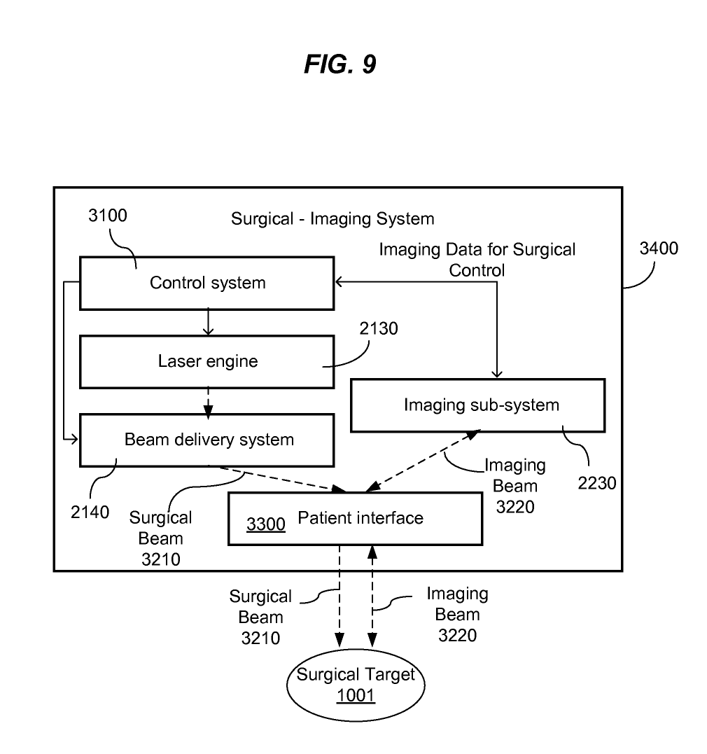

FIG. 9 shows an example of an imaging-guided laser surgical system with additional integration features. The imaging and surgical systems share a common patient interface 3300 which immobilizes target tissue 1001 (e.g., the eye) without having two separate patient interfaces as in FIG. 8. The surgical beam 3210 and the imaging beam 3220 are combined at the patient interface 3330 and are directed to the target 1001 by the common patient interface 3300. In addition, a common control module 3100 is provided to control both the imaging sub-system 2230 and the surgical part (the laser engine 2130 and the beam delivery system 2140). This increased integration between imaging and surgical parts allows accurate calibration of the two subsystems and the stability of the position of the patient and surgical volume. A common housing 3400 is provided to enclose both the surgical and imaging subsystems. When the two systems are not integrated into a common housing, the common patient interface 3300 can be part of either the imaging or the surgical subsystem.

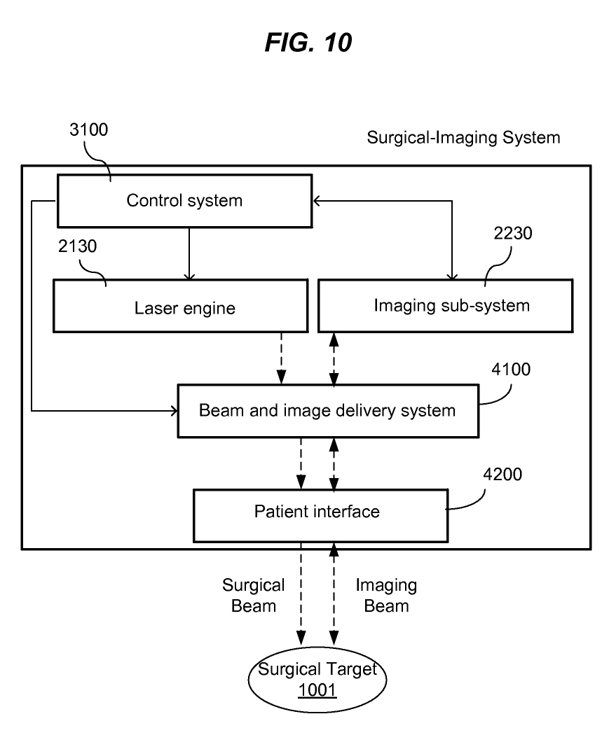

FIG. 10 shows an example of an imaging-guided laser surgical system where the laser surgical system and the imaging system share both a common beam delivery module 4100 and a common patient interface 4200. This integration further simplifies the system structure and system control operation.

In one implementation, the imaging system in the above and other examples can be an optical computed tomography (OCT) system and the laser surgical system is a femtosecond or picosecond laser based ophthalmic surgical system. In OCT, light from a low coherence, broadband light source such as a super luminescent diode is split into separate reference and signal beams. The signal beam is the imaging beam sent to the surgical target and the returned light of the imaging beam is collected and recombined coherently with the reference beam to form an interferometer. Scanning the signal beam perpendicularly to the optical axis of the optical train or the propagation direction of the light provides spatial resolution in the x-y direction while depth resolution comes from extracting differences between the path lengths of the reference arm and the returned signal beam in the signal arm of the interferometer. While the x-y scanner of different OCT implementations are essentially the same, comparing the path lengths and getting z-scan information can happen in different ways. In one implementation known as the time domain OCT, for example, the reference arm is continuously varied to change its path length while a photodetector detects interference modulation in the intensity of the re-combined beam. In a different implementation, the reference arm is essentially static and the spectrum of the combined light is analyzed for interference. The Fourier transform of the spectrum of the combined beam provides spatial information on the scattering from the interior of the sample. This method is known as the spectral domain or Fourier OCT method. In a different implementation known as a frequency swept OCT (S. R. Chinn, et. al., Opt. Lett. 22, 1997), a narrowband light source is used with its frequency swept rapidly across a spectral range. Interference between the reference and signal arms is detected by a fast detector and dynamic signal analyzer. An external cavity tuned diode laser or frequency tuned of frequency domain mode-locked (FDML) laser developed for this purpose (R. Huber et. Al. Opt. Express, 13, 2005) (S. H. Yun, IEEE J. of Sel. Q. El. 3(4) p. 1087-1096, 1997) can be used in these examples as a light source. A femtosecond laser used as a light source in an OCT system can have sufficient bandwidth and can provide additional benefits of increased signal to noise ratios.

The OCT imaging device in the systems in this document can be used to perform various imaging functions. For example, the OCT can be used to suppress complex conjugates resulting from the optical configuration of the system or the presence of the applanation plate, capture OCT images of selected locations inside the target tissue to provide three-dimensional positioning information for controlling focusing and scanning of the surgical laser beam inside the target tissue, or capture OCT images of selected locations on the surface of the target tissue or on the applanation plate to provide positioning registration for controlling changes in orientation that occur with positional changes of the target, such as from upright to supine. The OCT can be calibrated by a positioning registration process based on placement of marks or markers in one positional orientation of the target that can then be detected by the OCT module when the target is in another positional orientation. In other implementations, the OCT imaging system can be used to produce a probe light beam that is polarized to optically gather the information on the internal structure of the eye. The laser beam and the probe light beam may be polarized in different polarizations. The OCT can include a polarization control mechanism that controls the probe light used for said optical tomography to polarize in one polarization when traveling toward the eye and in a different polarization when traveling away from the eye. The polarization control mechanism can include, e.g., a wave-plate or a Faraday rotator.

The system in FIG. 10 is shown as a spectral OCT configuration and can be configured to share the focusing optics part of the beam delivery module between the surgical and the imaging systems. The main requirements for the optics are related to the operating wavelength, image quality, resolution, distortion etc. The laser surgical system can be a femtosecond laser system with a high numerical aperture system designed to achieve diffraction limited focal spot sizes, e.g., about 2 to 3 micrometers. Various femtosecond ophthalmic surgical lasers can operate at various wavelengths such as wavelengths of around 1.05 micrometer. The operating wavelength of the imaging device can be selected to be close to the laser wavelength so that the optics is chromatically compensated for both wavelengths. Such a system may include a third optical channel, a visual observation channel such as a surgical microscope, to provide an additional imaging device to capture images of the target tissue. If the optical path for this third optical channel shares optics with the surgical laser beam and the light of the OCT imaging device, the shared optics can be configured with chromatic compensation in the visible spectral band for the third optical channel and the spectral bands for the surgical laser beam and the OCT imaging beam.

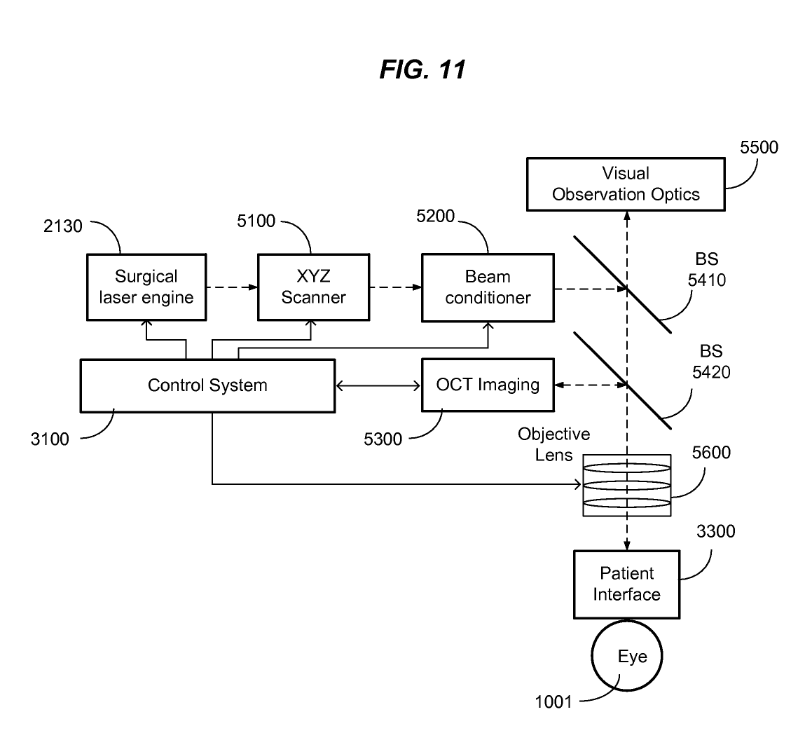

FIG. 11 shows a particular example of the design in FIG. 9 where the scanner 5100 for scanning the surgical laser beam and the beam conditioner 5200 for conditioning (collimating and focusing) the surgical laser beam are separate from the optics in the OCT imaging module 5300 for controlling the imaging beam for the OCT. The surgical and imaging systems share an objective lens 5600 module and the patient interface 3300. The objective lens 5600 directs and focuses both the surgical laser beam and the imaging beam to the patient interface 3300 and its focusing is controlled by the control module 3100. Two beam splitters 5410 and 5420 are provided to direct the surgical and imaging beams. The beam splitter 5420 is also used to direct the returned imaging beam back into the OCT imaging module 5300. Two beam splitters 5410 and 5420 also direct light from the target 1001 to a visual observation optics unit 5500 to provide direct view or image of the target 1001. The unit 5500 can be a lens imaging system for the surgeon to view the target 1001 or a camera to capture the image or video of the target 1001. Various beam splitters can be used, such as dichroic and polarization beam splitters, optical grating, holographic beam splitter or a combinations of these.

In some implementations, the optical components may be appropriately coated with antireflection coating for both the surgical and for the OCT wavelength to reduce glare from multiple surfaces of the optical beam path. Reflections would otherwise reduce the throughput of the system and reduce the signal to noise ratio by increasing background light in the OCT imaging unit. One way to reduce glare in the OCT is to rotate the polarization of the return light from the sample by wave-plate of Faraday isolator placed close to the target tissue and orient a polarizer in front of the OCT detector to preferentially detect light returned from the sample and suppress light scattered from the optical components.

In a laser surgical system, each of the surgical laser and the OCT system can have a beam scanner to cover the same surgical region in the target tissue. Hence, the beam scanning for the surgical laser beam and the beam scanning for the imaging beam can be integrated to share common scanning devices.

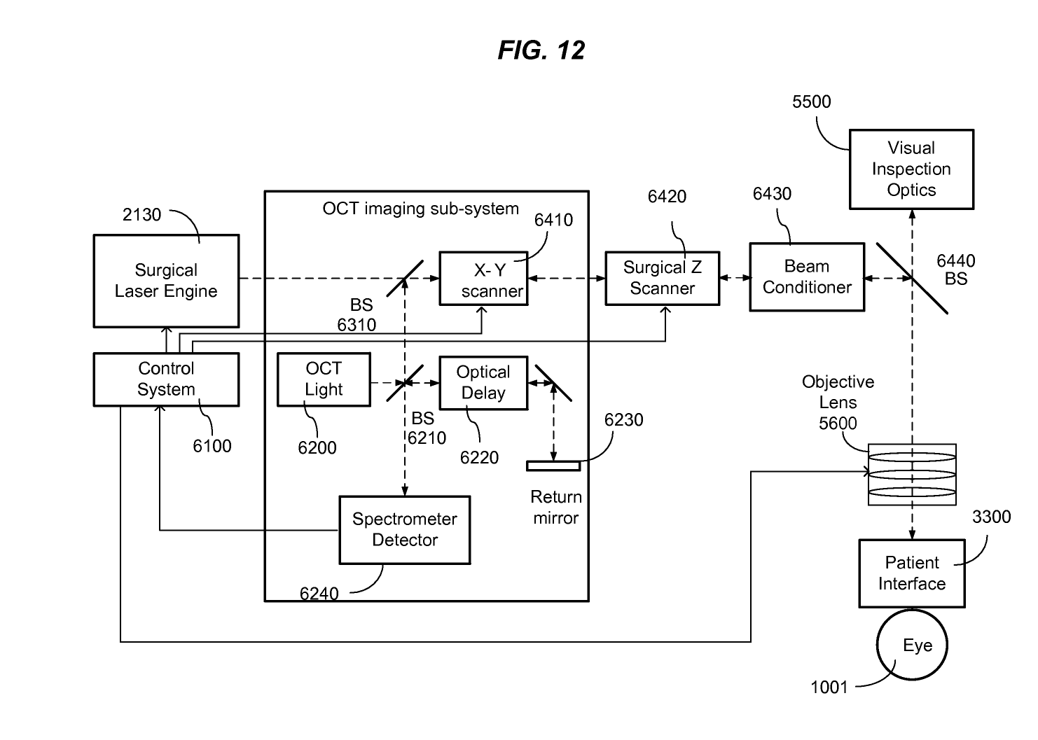

FIG. 12 shows an example of such a system in detail. In this implementation the x-y scanner 6410 and the z scanner 6420 are shared by both subsystems. A common control 6100 is provided to control the system operations for both surgical and imaging operations. The OCT sub-system includes an OCT light source 6200 that produce the imaging light that is split into an imaging beam and a reference beam by a beam splitter 6210. The imaging beam is combined with the surgical beam at the beam splitter 6310 to propagate along a common optical path leading to the target 1001. The scanners 6410 and 6420 and the beam conditioner unit 6430 are located downstream from the beam splitter 6310. A beam splitter 6440 is used to direct the imaging and surgical beams to the objective lens 5600 and the patient interface 3300.

In the OCT sub-system, the reference beam transmits through the beam splitter 6210 to an optical delay device 6220 and is reflected by a return mirror 6230. The returned imaging beam from the target 1001 is directed back to the beam splitter 6310 which reflects at least a portion of the returned imaging beam to the beam splitter 6210 where the reflected reference beam and the returned imaging beam overlap and interfere with each other. A spectrometer detector 6240 is used to detect the interference and to produce OCT images of the target 1001. The OCT image information is sent to the control system 6100 for controlling the surgical laser engine 2130, the scanners 6410 and 6420 and the objective lens 5600 to control the surgical laser beam. In one implementation, the optical delay device 6220 can be varied to change the optical delay to detect various depths in the target tissue 1001.

If the OCT system is a time domain system, the two subsystems use two different z-scanners because the two scanners operate in different ways. In this example, the z scanner of the surgical system operates by changing the divergence of the surgical beam in the beam conditioner unit without changing the path lengths of the beam in the surgical beam path. On the other hand, the time domain OCT scans the z-direction by physically changing the beam path by a variable delay or by moving the position of the reference beam return mirror. After calibration, the two z-scanners can be synchronized by the laser control module. The relationship between the two movements can be simplified to a linear or polynomial dependence, which the control module can handle or alternatively calibration points can define a look-up table to provide proper scaling. Spectral/Fourier domain and frequency swept source OCT devices have no z-scanner, the length of the reference arm is static. Besides reducing costs, cross calibration of the two systems will be relatively straightforward. There is no need to compensate for differences arising from image distortions in the focusing optics or from the differences of the scanners of the two systems since they are shared.

In practical implementations of the surgical systems, the focusing objective lens 5600 is slidably or movably mounted on a base and the weight of the objective lens is balanced to limit the force on the patient's eye. The patient interface 3300 can include an applanation lens attached to a patient interface mount. The patient interface mount is attached to a mounting unit, which holds the focusing objective lens. This mounting unit is designed to ensure a stable connection between the patient interface and the system in case of unavoidable movement of the patient and allows gentler docking of the patient interface onto the eye. Various implementations for the focusing objective lens can be used and one example is described in U.S. Pat. No. 5,336,215 to Hsueh. This presence of an adjustable focusing objective lens can change the optical path length of the optical probe light as part of the optical interferometer for the OCT sub-system. Movement of the objective lens 5600 and patient interface 3300 can change the path length differences between the reference beam and the imaging signal beam of the OCT in an uncontrolled way and this may degrade the OCT depth information detected by the OCT. This would happen not only in time-domain but also in spectral/Fourier domain and frequency-swept OCT systems.

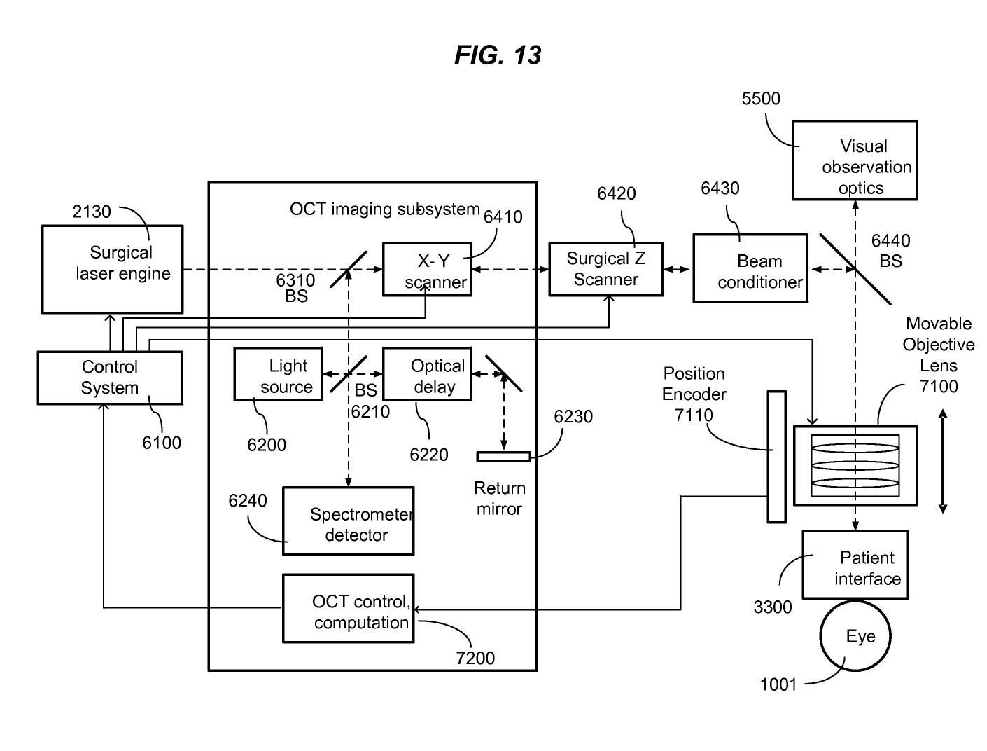

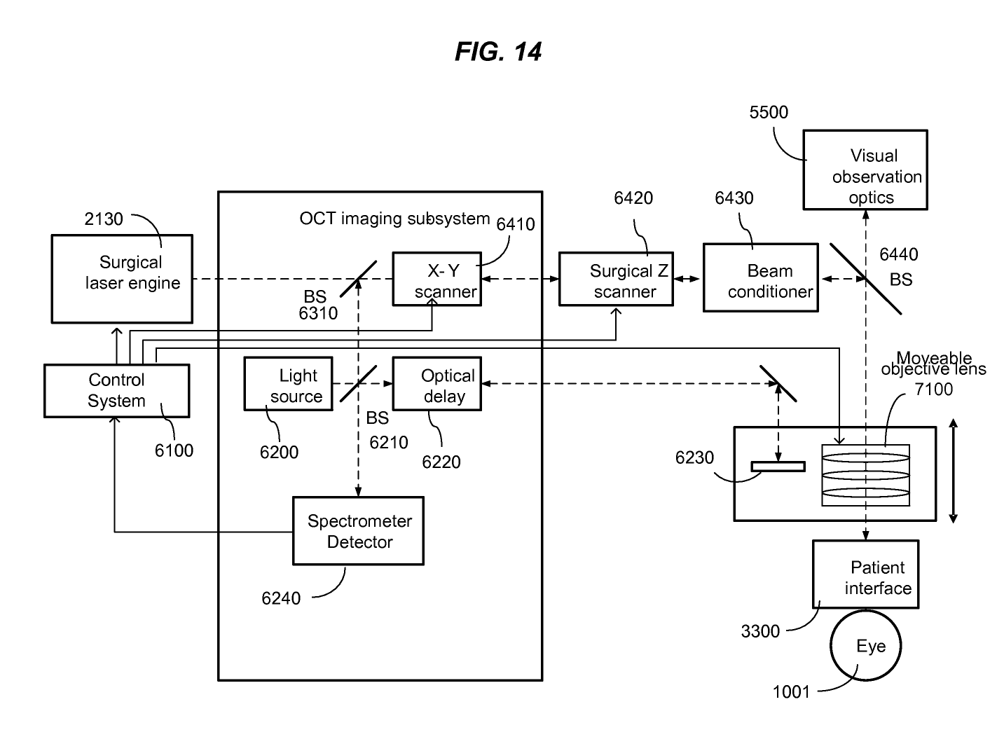

FIGS. 13 and 14 show exemplary imaging-guided laser surgical systems that address the technical issue associated with the adjustable focusing objective lens.

The system in FIG. 13 provides a position sensing device 7110 coupled to the movable focusing objective lens 7100 to measure the position of the objective lens 7100 on a slideable mount and communicates the measured position to a control module 7200 in the OCT system. The control system 6100 can control and move the position of the objective lens 7100 to adjust the optical path length traveled by the imaging signal beam for the OCT operation and the position of the lens 7100 is measured and monitored by the position encoder 7110 and direct fed to the OCT control 7200. The control module 7200 in the OCT system applies an algorithm, when assembling a 3D image in processing the OCT data, to compensate for differences between the reference arm and the signal arm of the interferometer inside the OCT caused by the movement of the focusing objective lens 7100 relative to the patient interface 3300. The proper amount of the change in the position of the lens 7100 computed by the OCT control module 7200 is sent to the control 6100 which controls the lens 7100 to change its position.

FIG. 14 shows another exemplary system where the return mirror 6230 in the reference arm of the interferometer of the OCT system or at least one part in an optical path length delay assembly of the OCT system is rigidly attached to the movable focusing objective lens 7100 so the signal arm and the reference arm undergo the same amount of change in the optical path length when the objective lens 7100 moves. As such, the movement of the objective lens 7100 on the slide is automatically compensated for path-length differences in the OCT system without additional need for a computational compensation.

The above examples for imaging-guided laser surgical systems, the laser surgical system and the OCT system use different light sources. In an even more complete integration between the laser surgical system and the OCT system, a femtosecond surgical laser as a light source for the surgical laser beam can also be used as the light source for the OCT system.

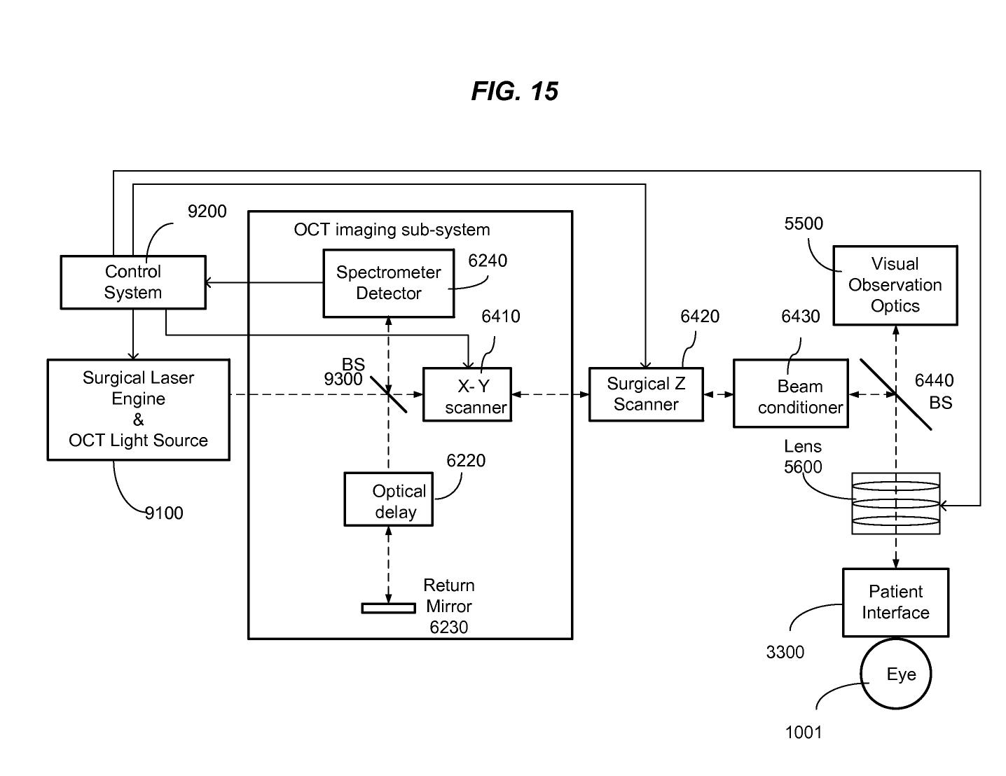

FIG. 15 shows an example where a femtosecond pulse laser in a light module 9100 is used to generate both the surgical laser beam for surgical operations and the probe light beam for OCT imaging. A beam splitter 9300 is provided to split the laser beam into a first beam as both the surgical laser beam and the signal beam for the OCT and a second beam as the reference beam for the OCT. The first beam is directed through an x-y scanner 6410 which scans the beam in the x and y directions perpendicular to the propagation direction of the first beam and a second scanner (z scanner) 6420 that changes the divergence of the beam to adjust the focusing of the first beam at the target tissue 1001. This first beam performs the surgical operations at the target tissue 1001 and a portion of this first beam is back scattered to the patient interface and is collected by the objective lens as the signal beam for the signal arm of the optical interferometer of the OCT system. This returned light is combined with the second beam that is reflected by a return mirror 6230 in the reference arm and is delayed by an adjustable optical delay element 6220 for a time-domain OCT to control the path difference between the signal and reference beams in imaging different depths of the target tissue 1001. The control system 9200 controls the system operations.

Surgical practice on the cornea has shown that a pulse duration of several hundred femtoseconds may be sufficient to achieve good surgical performance, while for OCT of a sufficient depth resolution broader spectral bandwidth generated by shorter pulses, e.g., below several tens of femtoseconds, are needed. In this context, the design of the OCT device dictates the duration of the pulses from the femtosecond surgical laser.

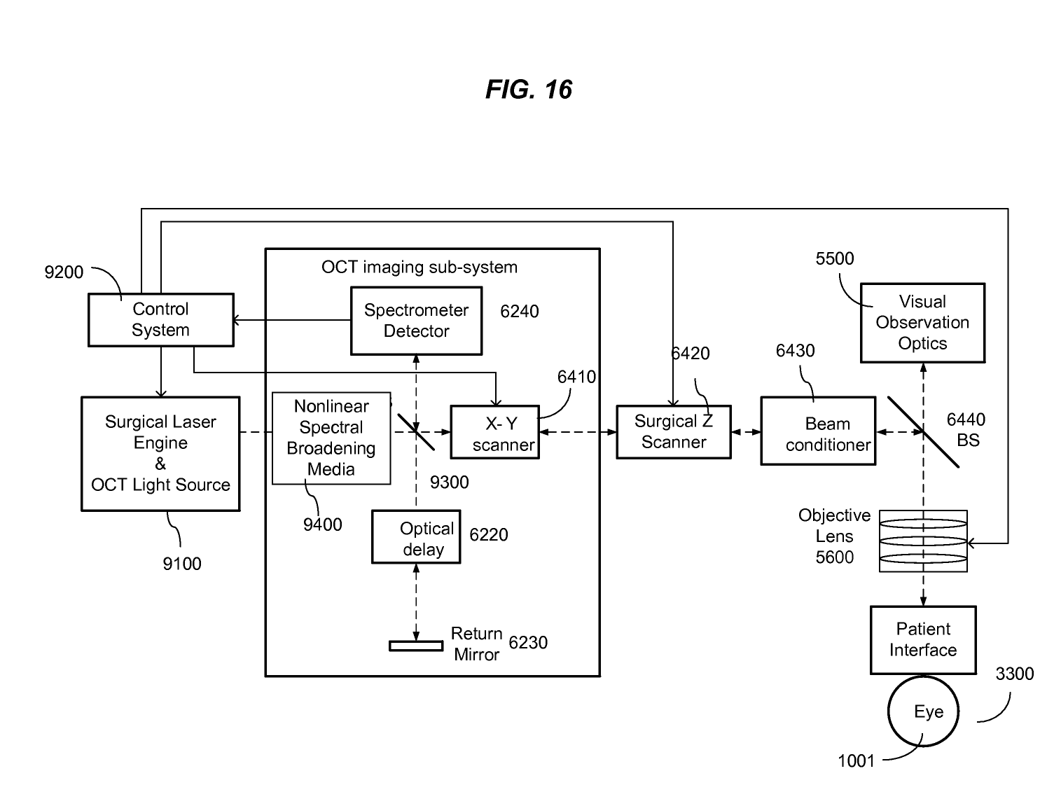

FIG. 16 shows another imaging-guided system that uses a single pulsed laser 9100 to produce the surgical light and the imaging light. A nonlinear spectral broadening media 9400 is placed in the output optical path of the femtosecond pulsed laser to use an optical non-linear process such as white light generation or spectral broadening to broaden the spectral bandwidth of the pulses from a laser source of relatively longer pulses, several hundred femtoseconds normally used in surgery. The media 9400 can be a fiber-optic material, for example. The light intensity requirements of the two systems are different and a mechanism to adjust beam intensities can be implemented to meet such requirements in the two systems. For example, beam steering mirrors, beam shutters or attenuators can be provided in the optical paths of the two systems to properly control the presence and intensity of the beam when taking an OCT image or performing surgery in order to protect the patient and sensitive instruments from excessive light intensity.

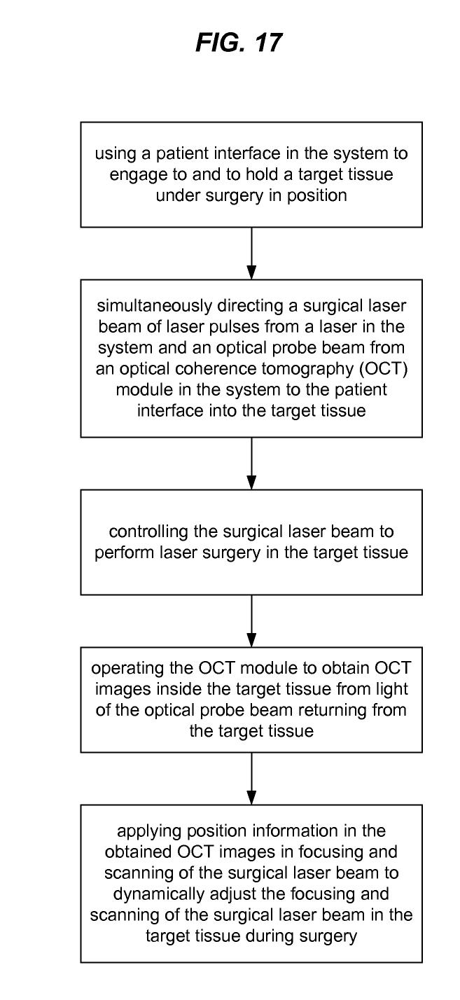

In operation, the above examples in FIGS. 8/16 can be used to perform imaging-guided laser surgery. FIG. 17 shows one example of a method for performing laser surgery by using an imaging-guided laser surgical system. This method uses a patient interface in the system to engage to and to hold a target tissue under surgery in position and simultaneously directs a surgical laser beam of laser pulses from a laser in the system and an optical probe beam from the OCT module in the system to the patient interface into the target tissue. The surgical laser beam is controlled to perform laser surgery in the target tissue and the OCT module is operated to obtain OCT images inside the target tissue from light of the optical probe beam returning from the target tissue. The position information in the obtained OCT images is applied in focusing and scanning of the surgical laser beam to adjust the focusing and scanning of the surgical laser beam in the target tissue before or during surgery.

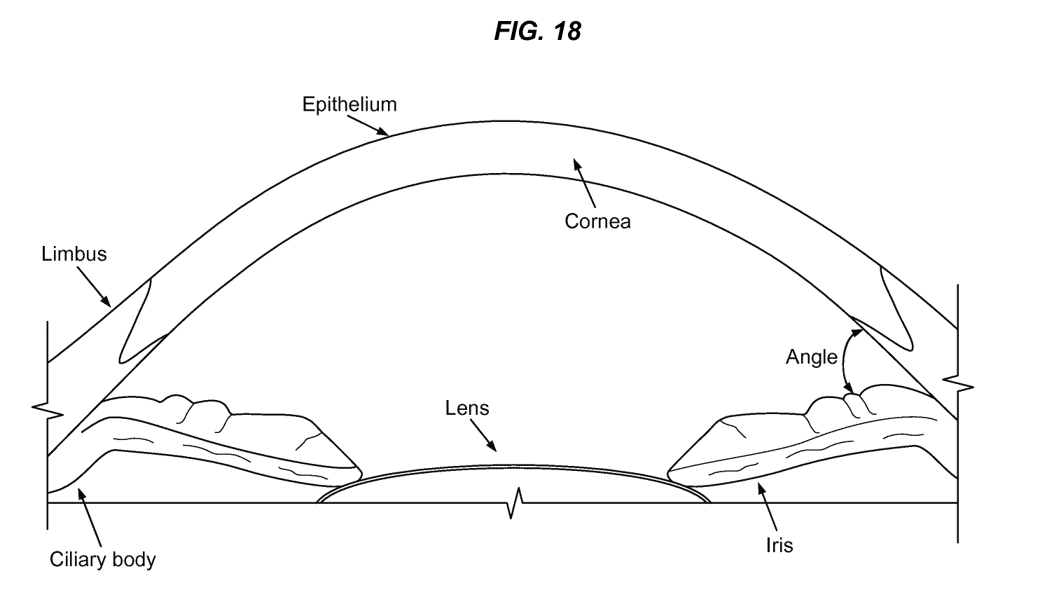

FIG. 18 shows an example of an OCT image of an eye. The contacting surface of the applanation lens in the patent interface can be configured to have a curvature that minimizes distortions or folds in the cornea due to the pressure exerted on the eye during applanation. After the eye is successfully applanated at the patient interface, an OCT image can be obtained. As illustrated in FIG. 18, the curvature of the lens and cornea as well as the distances between the lens and cornea are identifiable in the OCT image. Subtler features such as the epithelium-cornea interface are detectable. Each of these identifiable features may be used as an internal reference of the laser coordinates with the eye. The coordinates of the cornea and lens can be digitized using well-established computer vision algorithms such as Edge or Blob detection. Once the coordinates of the lens are established, they can be used to control the focusing and positioning of the surgical laser beam for the surgery.

Alternatively, a calibration sample material may be used to form a 3-D array of reference marks at locations with known position coordinates. The OCT image of the calibration sample material can be obtained to establish a mapping relationship between the known position coordinates of the reference marks and the OCT images of the reference marks in the obtained OCT image. This mapping relationship is stored as digital calibration data and is applied in controlling the focusing and scanning of the surgical laser beam during the surgery in the target tissue based on the OCT images of the target tissue obtained during the surgery. The OCT imaging system is used here as an example and this calibration can be applied to images obtained via other imaging techniques.

In an imaging-guided laser surgical system described here, the surgical laser can produce relatively high peak powers sufficient to drive strong field/multi-photon ionization inside of the eye (i.e. inside of the cornea and lens) under high numerical aperture focusing. Under these conditions, one pulse from the surgical laser generates a plasma within the focal volume. Cooling of the plasma results in a well defined damage zone or “bubble” that may be used as a reference point. The following sections describe a calibration procedure for calibrating the surgical laser against an OCT-based imaging system using the damage zones created by the surgical laser.

Before surgery can be performed, the OCT is calibrated against the surgical laser to establish a relative positioning relationship so that the surgical laser can be controlled in position at the target tissue with respect to the position associated with images in the OCT image of the target tissue obtained by the OCT. One way for performing this calibration uses a pre-calibrated target or “phantom” which can be damaged by the laser as well as imaged with the OCT. The phantom can be fabricated from various materials such as a glass or hard plastic (e.g. PMMA) such that the material can permanently record optical damage created by the surgical laser. The phantom can also be selected to have optical or other properties (such as water content) that are similar to the surgical target.

The phantom can be, e.g., a cylindrical material having a diameter of at least 10 mm (or that of the scanning range of the delivery system) and a cylindrical length of at least 10 mm long spanning the distance of the epithelium to the crystalline lens of the eye, or as long as the scanning depth of the surgical system. The upper surface of the phantom can be curved to mate seamlessly with the patient interface or the phantom material may be compressible to allow full applanation. The phantom may have a three dimensional grid such that both the laser position (in x and y) and focus (z), as well as the OCT image can be referenced against the phantom.

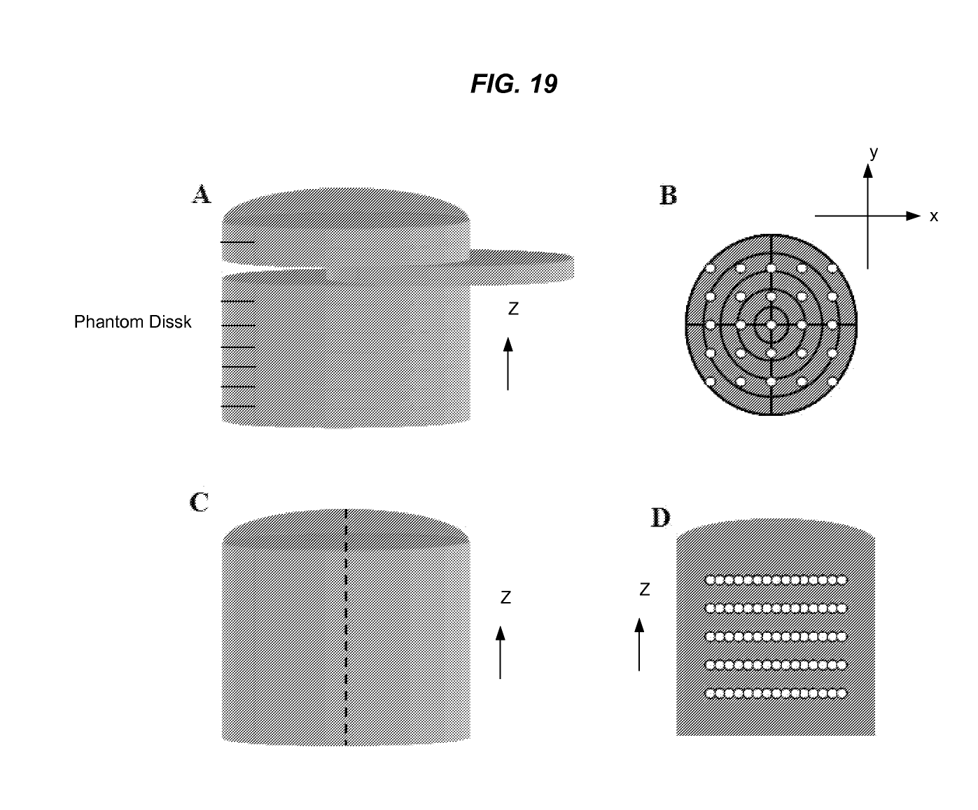

FIGS. 19A-19D illustrate two exemplary configurations for the phantom. FIG. 19A illustrates a phantom that is segmented into thin disks. FIG. 19B shows a single disk patterned to have a grid of reference marks as a reference for determining the laser position across the phantom (i.e. the x- and y-coordinates). The z-coordinate (depth) can be determined by removing an individual disk from the stack and imaging it under a confocal microscope.

FIG. 19C illustrates a phantom that can be separated into two halves. Similar to the segmented phantom in FIG. 19A, this phantom is structured to contain a grid of reference marks as a reference for determining the laser position in the x- and y-coordinates. Depth information can be extracted by separating the phantom into the two halves and measuring the distance between damage zones. The combined information can provide the parameters for image guided surgery.

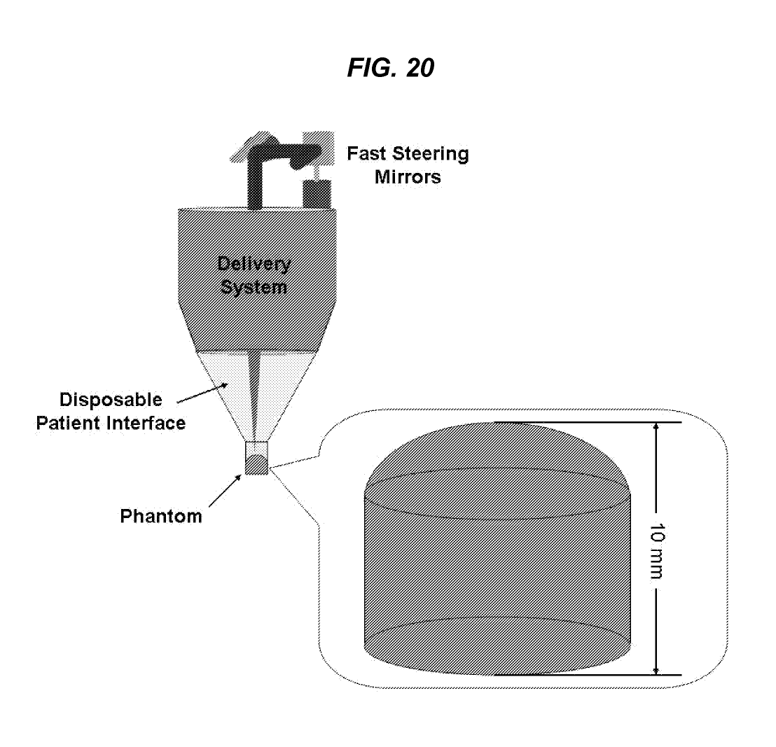

FIG. 20 shows a surgical system part of the imaging-guided laser surgical system. This system includes steering mirrors which may be actuated by actuators such as galvanometers or voice coils, an objective lens e and a disposable patient interface. The surgical laser beam is reflected from the steering mirrors through the objective lens. The objective lens focuses the beam just after the patient interface. Scanning in the x- and y-coordinates is performed by changing the angle of the beam relative to the objective lens. Scanning in z-plane is accomplished by changing the divergence of the incoming beam using a system of lens upstream to the steering mirrors.

In this example, the conical section of the disposable patient interface may be either air spaced or solid and the section interfacing with the patient includes a curved contact lens. The curved contact lens can be fabricated from fused silica or other material resistant to forming color centers when irradiated with ionizing radiation. The radius of curvature is on the upper limit of what is compatible with the eye, e.g., about 10 mm.

The first step in the calibration procedure is docking the patient interface with the phantom. The curvature of the phantom matches the curvature of the patient interface. After docking, the next step in the procedure involves creating optical damage inside of the phantom to produce the reference marks.

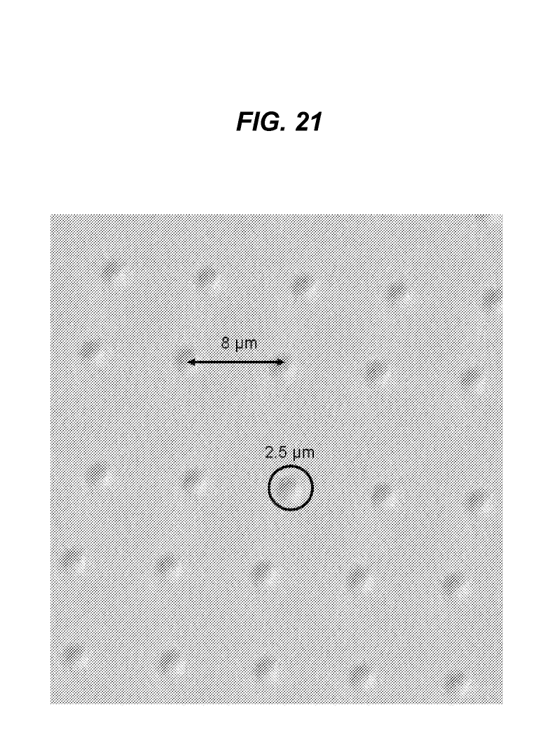

FIG. 21 shows examples of actual damage zones produced by a femtosecond laser in glass. The separation between the damage zones is on average 8 μm (the pulse energy is 2.2 μJ with duration of 580 fs at full width at half maximum). The optical damage depicted in FIG. 21 shows that the damage zones created by the femtosecond laser are well-defined and discrete. In the example shown, the damage zones have a diameter of about 2.5 μm. Optical damage zones similar to that shown in FIG. 20 are created in the phantom at various depths to form a 3-D array of the reference marks. These damage zones are referenced against the calibrated phantom either by extracting the appropriate disks and imaging it under a confocal microscope (FIG. 19A) or by splitting the phantom into two halves and measuring the depth using a micrometer (FIG. 19C). The x- and y-coordinates can be established from the pre-calibrated grid.

After damaging the phantom with the surgical laser, OCT on the phantom is performed. The OCT imaging system provides a 3D rendering of the phantom establishing a relationship between the OCT coordinate system and the phantom. The damage zones are detectable with the imaging system. The OCT and laser may be cross-calibrated using the phantom's internal standard. After the OCT and the laser are referenced against each other, the phantom can be discarded.

Prior to surgery, the calibration can be verified. This verification step involves creating optical damage at various positions inside of a second phantom. The optical damage should be intense enough such that the multiple damage zones which create a circular pattern can be imaged by the OCT. After the pattern is created, the second phantom is imaged with the OCT. Comparison of the OCT image with the laser coordinates provides the final check of the system calibration prior to surgery.

Once the coordinates are fed into the laser, laser surgery can be performed inside the eye. This involves photo-emulsification of the lens using the laser, as well as other laser treatments to the eye. The surgery can be stopped at any time and the anterior segment of the eye (FIG. 17) can be re-imaged to monitor the progress of the surgery; moreover, after the IOL (intra ocular lens) is inserted, imaging the IOL (with light or no applanation) provides information regarding the position of the IOL in the eye. This information may be utilized by the physician to refine the position of the IOL.

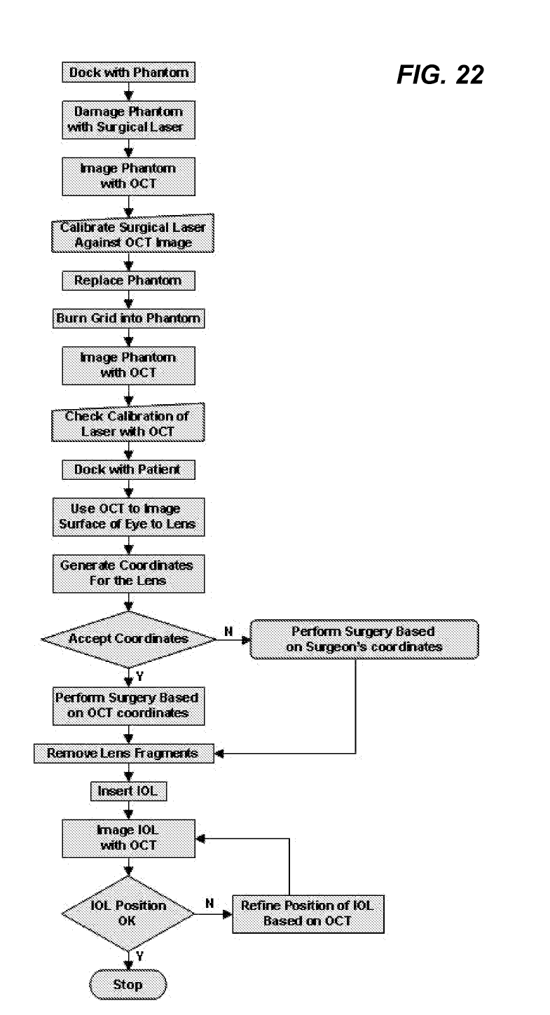

FIG. 22 shows an example of the calibration process and the post-calibration surgical operation. This examples illustrates a method for performing laser surgery by using an imaging-guided laser surgical system can include using a patient interface in the system, that is engaged to hold a target tissue under surgery in position, to hold a calibration sample material during a calibration process before performing a surgery; directing a surgical laser beam of laser pulses from a laser in the system to the patient interface into the calibration sample material to burn reference marks at selected three-dimensional reference locations; directing an optical probe beam from an optical coherence tomography (OCT) module in the system to the patient interface into the calibration sample material to capture OCT images of the burnt reference marks; and establishing a relationship between positioning coordinates of the OCT module and the burnt reference marks. After the establishing the relationship, a patient interface in the system is used to engage to and to hold a target tissue under surgery in position. The surgical laser beam of laser pulses and the optical probe beam are directed to the patient interface into the target tissue. The surgical laser beam is controlled to perform laser surgery in the target tissue. The OCT module is operated to obtain OCT images inside the target tissue from light of the optical probe beam returning from the target tissue and the position information in the obtained OCT images and the established relationship are applied in focusing and scanning of the surgical laser beam to adjust the focusing and scanning of the surgical laser beam in the target tissue during surgery. While such calibrations can be performed immediately prior to laser surgery, they can also be performed at various intervals before a procedure, using calibration validations that demonstrated a lack of drift or change in calibration during such intervals.

The following examples describe imaging-guided laser surgical techniques and systems that use images of laser-induced photodisruption byproducts for alignment of the surgical laser beam.

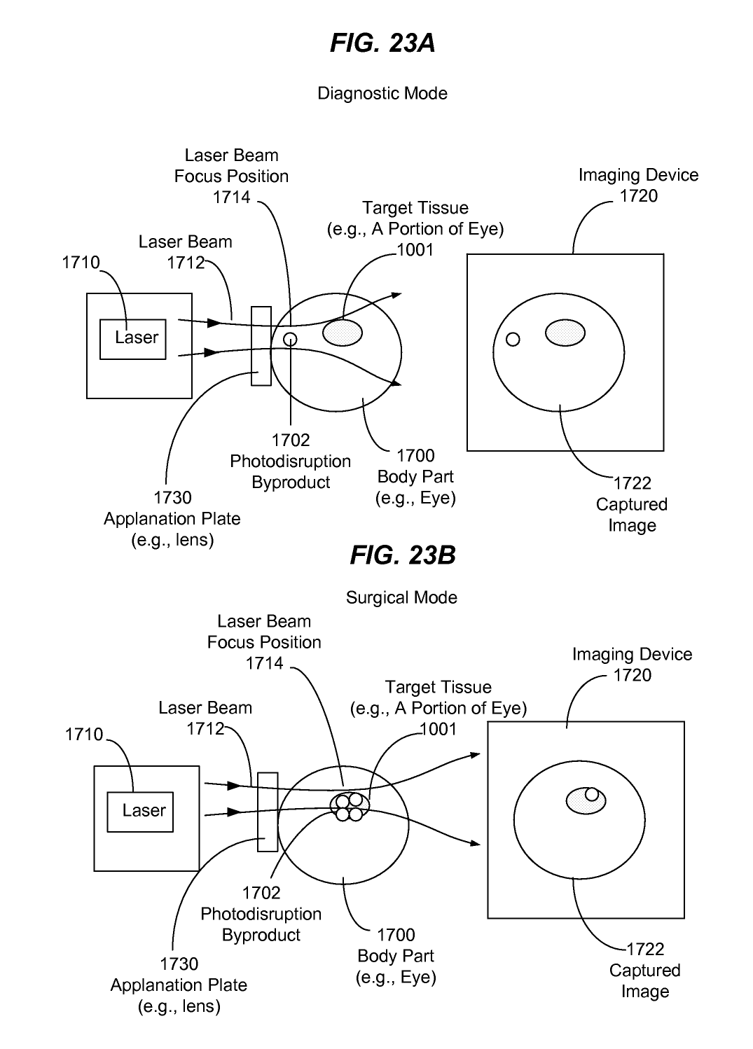

FIGS. 23A and 23B illustrate another implementation of the present technique in which actual photodisruption byproducts in the target tissue are used to guide further laser placement. A pulsed laser 1710, such as a femtosecond or picosecond laser, is used to produce a laser beam 1712 with laser pulses to cause photodisruption in a target tissue 1001. The target tissue 1001 may be a part of a body part 1700 of a subject, e.g., a portion of the lens of one eye. The laser beam 1712 is focused and directed by an optics module for the laser 1710 to a target tissue position in the target tissue 1001 to achieve a certain surgical effect. The target surface is optically coupled to the laser optics module by an applanation plate 1730 that transmits the laser wavelength, as well as image wavelengths from the target tissue. The applanation plate 1730 can be an applanation lens. An imaging device 1720 is provided to collect reflected or scattered light or sound from the target tissue 1001 to capture images of the target tissue 1001 either before or after (or both) the applanation plate is applied. The captured imaging data is then processed by the laser system control module to determine the desired target tissue position. The laser system control module moves or adjusts optical or laser elements based on standard optical models to ensure that the center of photodisruption byproduct 1702 overlaps with the target tissue position. This can be a dynamic alignment process where the images of the photodisruption byproduct 1702 and the target tissue 1001 are continuously monitored during the surgical process to ensure that the laser beam is properly positioned at each target tissue position.

In one implementation, the laser system can be operated in two modes: first in a diagnostic mode in which the laser beam 1712 is initially aligned by using alignment laser pulses to create photodisruption byproduct 1702 for alignment and then in a surgical mode where surgical laser pulses are generated to perform the actual surgical operation. In both modes, the images of the disruption byproduct 1702 and the target tissue 1001 are monitored to control the beam alignment. FIG. 17A shows the diagnostic mode where the alignment laser pulses in the laser beam 1712 may be set at a different energy level than the energy level of the surgical laser pulses. For example, the alignment laser pulses may be less energetic than the surgical laser pulses but sufficient to cause significant photodisruption in the tissue to capture the photodisruption byproduct 1702 at the imaging device 1720. The resolution of this coarse targeting may not be sufficient to provide desired surgical effect. Based on the captured images, the laser beam 1712 can be aligned properly. After this initial alignment, the laser 1710 can be controlled to produce the surgical laser pulses at a higher energy level to perform the surgery. Because the surgical laser pulses are at a different energy level than the alignment laser pulses, the nonlinear effects in the tissue material in the photodisruption can cause the laser beam 1712 to be focused at a different position from the beam position during the diagnostic mode. Therefore, the alignment achieved during the diagnostic mode is a coarse alignment and additional alignment can be further performed to precisely position each surgical laser pulse during the surgical mode when the surgical laser pulses perform the actual surgery. Referring to FIG. 23A, the imaging device 1720 captures the images from the target tissue 1001 during the surgical mode and the laser control module adjust the laser beam 1712 to place the focus position 1714 of the laser beam 1712 onto the desired target tissue position in the target tissue 1001. This process is performed for each target tissue position.

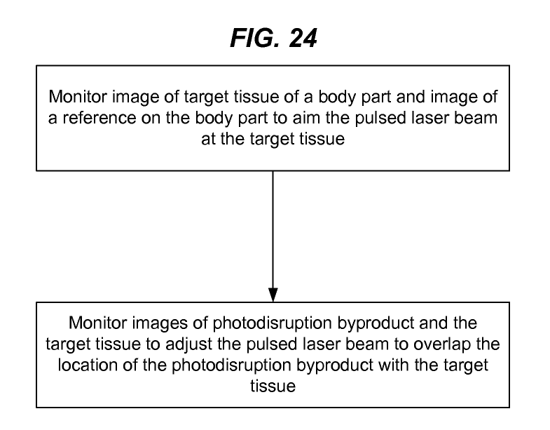

FIG. 24 shows one implementation of the laser alignment where the laser beam is first approximately aimed at the target tissue and then the image of the photodisruption byproduct is captured and used to align the laser beam. The image of the target tissue of the body part as the target tissue and the image of a reference on the body part are monitored to aim the pulsed laser beam at the target tissue. The images of photodisruption byproduct and the target tissue are used to adjust the pulsed laser beam to overlap the location of the photodisruption byproduct with the target tissue.

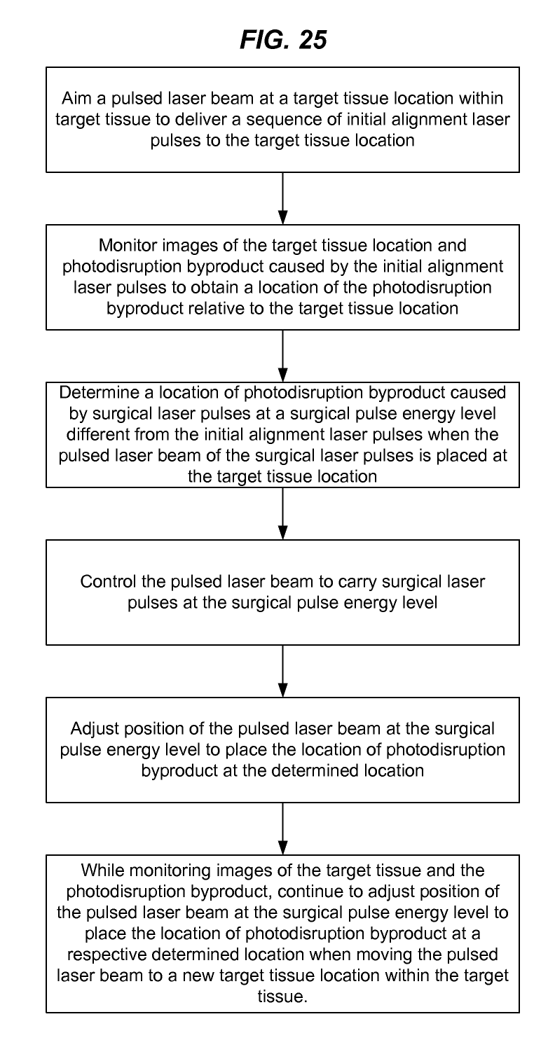

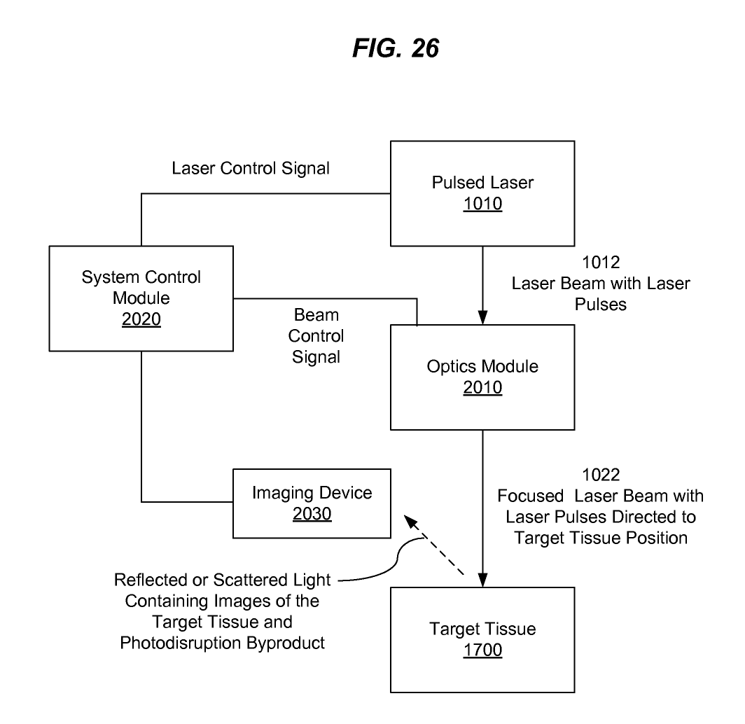

FIG. 25 shows one implementation of the laser alignment method based on imaging photodisruption byproduct in the target tissue in laser surgery. In this method, a pulsed laser beam is aimed at a target tissue location within target tissue to deliver a sequence of initial alignment laser pulses to the target tissue location. The images of the target tissue location and photodisruption byproduct caused by the initial alignment laser pulses are monitored to obtain a location of the photodisruption byproduct relative to the target tissue location. The location of photodisruption byproduct caused by surgical laser pulses at a surgical pulse energy level different from the initial alignment laser pulses is determined when the pulsed laser beam of the surgical laser pulses is placed at the target tissue location. The pulsed laser beam is controlled to carry surgical laser pulses at the surgical pulse energy level. The position of the pulsed laser beam is adjusted at the surgical pulse energy level to place the location of photodisruption byproduct at the determined location. While monitoring images of the target tissue and the photodisruption byproduct, the position of the pulsed laser beam at the surgical pulse energy level is adjusted to place the location of photodisruption byproduct at a respective determined location when moving the pulsed laser beam to a new target tissue location within the target tissue.

FIG. 26 shows an exemplary laser surgical system based on the laser alignment using the image of the photodisruption byproduct. An optics module 2010 is provided to focus and direct the laser beam to the target tissue 1700. The optics module 2010 can include one or more lenses and may further include one or more reflectors. A control actuator is included in the optics module 2010 to adjust the focusing and the beam direction in response to a beam control signal. A system control module 2020 is provided to control both the pulsed laser 1010 via a laser control signal and the optics module 2010 via the beam control signal. The system control module 2020 processes image data from the imaging device 2030 that includes the position offset information for the photodisruption byproduct 1702 from the target tissue position in the target tissue 1700. Based on the information obtained from the image, the beam control signal is generated to control the optics module 2010 which adjusts the laser beam. A digital processing unit is included in the system control module 2020 to perform various data processing for the laser alignment.

The imaging device 2030 can be implemented in various forms, including an optical coherent tomography (OCT) device. In addition, an ultrasound imaging device can also be used. The position of the laser focus is moved so as to place it grossly located at the target at the resolution of the imaging device. The error in the referencing of the laser focus to the target and possible non-linear optical effects such as self focusing that make it difficult to accurately predict the location of the laser focus and subsequent photodisruption event. Various calibration methods, including the use of a model system or software program to predict focusing of the laser inside a material can be used to get a coarse targeting of the laser within the imaged tissue. The imaging of the target can be performed both before and after the photodisruption. The position of the photodisruption by products relative to the target is used to shift the focal point of the laser to better localize the laser focus and photodisruption process at or relative to the target. Thus the actual photodisruption event is used to provide a precise targeting for the placement of subsequent surgical pulses.

Photodisruption for targeting during the diagnostic mode can be performed at a lower, higher or the same energy level that is required for the later surgical processing in the surgical mode of the system. A calibration may be used to correlate the localization of the photodisruptive event performed at a different energy in diagnostic mode with the predicted localization at the surgical energy because the optical pulse energy level can affect the exact location of the photodisruptive event. Once this initial localization and alignment is performed, a volume or pattern of laser pulses (or a single pulse) can be delivered relative to this positioning. Additional sampling images can be made during the course of delivering the additional laser pulses to ensure proper localization of the laser (the sampling images may be obtained with use of lower, higher or the same energy pulses). In one implementation, an ultrasound device is used to detect the cavitation bubble or shock wave or other photodisruption byproduct. The localization of this can then be correlated with imaging of the target, obtained via ultrasound or other modality. In another embodiment, the imaging device is simply a biomicroscope or other optical visualization of the photodisruption event by the operator, such as optical coherence tomography. With the initial observation, the laser focus is moved to the desired target position, after which a pattern or volume of pulses is delivered relative to this initial position.

As a specific example, a laser system for precise subsurface photodisruption can include means for generating laser pulses capable of generating photodisruption at repetition rates of 100-1000 Million pulses per second, means for coarsely focusing laser pulses to a target below a surface using an image of the target and a calibration of the laser focus to that image without creating a surgical effect, means for detecting or visualizing below a surface to provide an image or visualization of a target the adjacent space or material around the target and the byproducts of at least one photodisruptive event coarsely localized near the target, means for correlating the position of the byproducts of photodisruption with that of the sub surface target at least once and moving the focus of the laser pulse to position the byproducts of photodisruption at the sub surface target or at a relative position relative to the target, means for delivering a subsequent train of at least one additional laser pulse in pattern relative to the position indicated by the above fine correlation of the byproducts of photodisruption with that of the sub surface target, and means for continuing to monitor the photodisruptive events during placement of the subsequent train of pulses to further fine tune the position of the subsequent laser pulses relative to the same or revised target being imaged.

The above techniques and systems can be used deliver high repetition rate laser pulses to subsurface targets with a precision required for contiguous pulse placement, as needed for cutting or volume disruption applications. This can be accomplished with or without the use of a reference source on the surface of the target and can take into account movement of the target following applanation or during placement of laser pulses.

While this specification contains many specifics, these should not be construed as limitations on the scope of any invention or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

1. A laser treatment method for treating a lens of an eye, comprising:
defining a target boundary of a target region in the lens;
applying surgical laser pulses to the target boundary, effectively resulting in a separation of the target region from the rest of the lens; and
removing the separated target region from the lens.

2. The method of claim 1, wherein the defining the target boundary comprises determining at least one of:
a transparency of a lens region;
an optical density of a lens region;
a refractive error of the lens irrespective of the source of the refractive error;
a reduced accommodation of a lens region;
an image of a lens region;
a flexibility of a lens region; and
individual or normative data of the lens.

3. The method of claim 1, wherein the defining the target boundary comprises:
generating probe bubbles in the lens; and
identifying a boundary separating two regions wherein a mechanical or optical characteristic of the probe bubbles is different in the two regions.

4. The method of claim 1, wherein the defining the target boundary comprises:
applying marker laser pulses to outline the target boundary.

5. The method of claim 4, comprising:
applying the marker laser pulses by a laser source using marker pulse settings; and
applying the surgical laser pulses by the same laser source using surgical pulse settings.

6. The method of claim 4, wherein the defining the target boundary comprises:
applying marker laser pulses to outline the target boundary; and
imaging the outlined target boundary in an iterative sequence.

7. The method of claim 1, wherein the defining the target boundary comprises:
outlining the target boundary by a non-laser plasma source.

8. The method of claim 1, wherein the defining the target boundary comprises at least one of:
defining the target region in a central region of a nucleus of the eye; and
defining the target region in a peripheral region of the nucleus of the eye.

9. The method of claim 1, wherein the defining the target boundary comprises at least one of:
defining the target region in a session separate from a session when the surgical laser pulses are applied; and
defining the target region in the same session when the surgical laser pulses are applied.

10. The method of claim 1, wherein the applying the surgical pulses comprises:
applying surgical laser pulses with at least one of:
a separation of generated surgical bubbles between 1 micron and 50 microns;
a duration of the surgical laser pulses between 0.01 picoseconds and 50 picoseconds;
an energy per surgical laser pulse between 0.5 μJ and 50 μJ; and
a surgical laser pulse repetition rate between 10 kHz and 100 MHz.

11. The method of claim 1, wherein the applying the surgical pulses comprises:
applying surgical laser pulses with settings between
a lower threshold, selected based on the surgical laser pulses achieving a desired result; and
an upper threshold, selected based on the surgical laser pulses avoiding a damage to a selected tissue.

12. The method of claim 1, wherein the applying the surgical laser pulses comprises:
applying the surgical laser pulses to a posterior region of the target boundary; and
applying the surgical laser pulses to an anterior region of the target boundary after the application of the surgical laser pulses to a posterior region of the target boundary.

13. The method of claim 1, wherein
the defining the target boundary and the applying surgical laser pulses is performed before making an incision on the eye.

14. The method of claim 1, wherein the removing the separated target region comprises:
fragmenting a portion of the target region prior to the removal from the lens.

15. The method of claim 14, wherein the fragmenting of the separated target region comprises:
fragmenting a portion of the target region by at least one of a photodisruption, a use of ultrasound, and a use of heated fluids.

16. The method of claim 14, wherein the applying the surgical laser pulses and the fragmenting the target region is performed in a combined manner, comprising the steps of:
applying the surgical laser pulses to a posterior region of the target boundary;
applying fragmenting laser pulses to the target region for fragmenting a portion of the target region after applying the surgical laser pulses to a posterior region of the target boundary; and
applying the surgical laser pulses to an anterior region of the target boundary after applying the fragmenting laser pulses to a posterior region of the target boundary.

17. The method of claim 1, wherein the removing the separated target region comprises:
forming an opening in the lens.

18. The method of claim 17, wherein the forming the opening comprises:
forming the opening with one of a photodisruption, an ultrasound-based method, a heated fluid-based method and a mechanical surgical method.

19. The method of claim 17, wherein the removing the separated target region comprises:
fragmenting the target region; and
aspirating the fragmented target region through the opening.

20. The method of claim 1, further comprising:
introducing a pharmacological agent, a medication, a fluid or an implantable device in a void left behind by the removed target.

21. A method for restoring physiologic functioning of a lens, comprising:
identifying a volume of lens tissue to be removed;
separating the identified volume of lens tissue from the surrounding lens tissue by laser-fragmenting a boundary of the identified volume of the lens tissue;
removing the identified volume of lens tissue from the lens; and
managing a reapproximation of a remaining lens portion to improve the functionality of the lens.

22. The method of claim 21, wherein the lens tissue to be removed is identified by at least one of:
comparing a size of the lens to normative data or to other ocular structures;
determining a measure of lens flexibility or eye accommodation;
determining a reduction of an optical transparency; and
determining a refractive error.

23. The method as in claim 21, wherein the separating the identified volume of lens tissue comprises:
outlining the boundary of the identified volume by marker laser pulses; and
separating the boundary of the identified volume by surgical laser pulses.

24. The method of claim 21, wherein the removing the identified volume of lens tissue comprises:
forming an opening in the lens; and
making corresponding incisions in a cornea of the eye.

25. The method of claim 21, wherein the managing the reapproximation of the remaining lens portion comprises at least one of:
infusing at least one of a medication and a fluid into a void left by the removal of the identified volume; and
inserting an implantable device in the into a void left by the removal of the identified volume.

26. A laser surgical device for a treatment of a lens in an eye, comprising:
an imaging module, configured to image the lens to provide information for defining a target boundary of a target region in the lens for treatment;
a surgical laser module, configured to apply surgical laser pulses to the target boundary effectively resulting in a separation of the target region from the rest of the lens; and
a surgical intervention module, configured to remove the separated target region from the lens.

27. The device as in claim 26, wherein the imaging module comprises an optical coherence tomography (OCT) imaging module.

28. The device as in claim 26, comprising:
means for defining the target boundary comprises determining at least one of:
a transparency of a lens region;
an optical density of a lens region;
a refractive error of the lens irrespective of the source of the refractive error;
a reduced accommodation of a lens region;
an image of a lens region;
a flexibility of a lens region; and
individual or normative data of the lens.
